(19) United States
(12) Patent Application Publication
(10) Pub. No.: US 2009/0163715 A1
(43) Pub. Date: Jun. 25, 2009

(54) Intermediate in Production of [2-(3,3,5,5-Tetramethylcyclohexyl)phenyl]Piperazine Compound

(75) Inventors: Mitsuo Nagai, Ibaraki (JP); Kazumasa Nara, Ibaraki (JP); Kazunori Wakasugi, Ibaraki (JP); Toshihiko Naito, Ibaraki (JP)

Correspondence Address: BIRCH STEWART KOLASCH & BIRCH, PO BOX 747, FALLS CHURCH, VA, 22040-0747, US

(73) Assignee: EISAI R&D MANAGEMENT CO., LTD., Tokyo (JP)

(21) Appl. No.: 11/920,920

(22) PCT Filed: May 25, 2006
(86) PCT No.: PCT/JP2006/310450

§ 371 Date: Dec. 21, 2008

(30) Foreign Application Priority Data
May 25, 2005 (JP) 2005-151697
Dec. 16, 2005 (JP) PCT/JP2005/023166

(51) Int. Cl. C07D 241/04; C07C 211/44; C07D 207/30; C07D 211/08; C07C 55/06; C07C 57/15; C07C 309/01

(52) U.S. Cl. 544/403; 564/305; 548/563; 546/192; 562/597; 562/595; 562/41

(57) ABSTRACT

A compound represented by the general formula below or a salt thereof is a useful intermediate in the production of a [2-(3,3,5,5-tetramethylcyclohexyl)phenyl]piperazine compound that has excellent cell adhesion inhibitory effect and cell invasion inhibitory effect and therefore is useful as a therapeutic or prophylactic agent for various inflammatory diseases and autoimmune diseases induced by the adhesion and invasion of leukocytes, such as inflammatory bowel diseases (particularly ulcerative colitis or Crohn's disease), irritable bowel syndrome, rheumatoid arthritis, psoriasis, multiple sclerosis, asthma and atopic dermatitis.

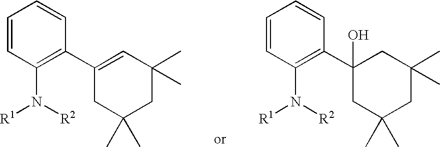

wherein R¹ and R² independently represent hydrogen or a protecting group for an amino group, or R¹ and R² bond together to form an optionally substituted pyrrole ring.

### TECHNICAL FIELD

Some of [2-(3,3,5,5-tetramethylcyclohexyl)phenyl]piperazine compounds have cell adhesion inhibitory action or cell infiltration inhibitory action, and the present invention relates to useful intermediates in production of the [2-(3,3,5,5-tetramethylcyclohexyl)phenyl]piperazine compounds or pharmacologically acceptable salts thereof.

### BACKGROUND ART

Inflammatory reaction is accompanied by infiltration of leukocytes, typically neutrophils and lymphocytes, into inflammatory sites.

Infiltration of leukocytes is defined as migration of leukocytes such as neutrophils and lymphocytes out of vessels and into the surrounding tissues as a consequence of initiation and activation by cytokines, chemokines, lipids and complement to interact called “rolling” or “tethering” with vascular endothelial cells activated by cytokines such as IL-1 or TNFα, followed by adhesion to the vascular endothelial cells.

As explained below, relationship between leukocyte adhesion or infiltration and various inflammatory diseases and autoimmune diseases was reported. Such reports have raised the possibility that compounds having cell adhesion inhibitory action or cell infiltration inhibitory action may serve as therapeutic or prophylactic agents for such diseases.

(1) Therapeutic or prophylactic agents for inflammatory bowel disease (ulcerative colitis, Crohn's disease and the like) (see Non-patent documents 1, 2 and 3)
(2) Therapeutic or prophylactic agents for irritable bowel syndrome (see Non-patent document 4)

[Non-patent document 1] Inflammatory Bowel Disease (N. Engl. J. Med., 347: 417-429 (2002))

[Non-patent document 2] Natalizumab for active Crohn's disease (N. Engl. J. Med., 348: 24-32 (2003))

[Non-patent document 3] Granulocyte adsorption therapy in active period of ulcerative colitis (Japanese Journal of Apheresis 18: 117-131 (1999))

[Non-patent document 4] A role for inflammation in irritable bowel syndrome (Gut., 51: i41-i44 (2002))

### DISCLOSURE OF THE INVENTION

### Problems to be Solved by the Invention

[2-(3,3,5,5-Tetramethylcyclohexyl)phenyl]piperazine compounds are novel compounds having excellent cell adhesion inhibitory action and cell infiltration inhibitory action, which are useful as therapeutic or prophylactic agents for various inflammatory diseases and autoimmune diseases associated with adhesion and infiltration of leukocytes, such as inflammatory bowel disease (particularly ulcerative colitis or Crohn's disease), irritable bowel syndrome, rheumatoid arthritis, psoriasis, multiple sclerosis, asthma and atopic dermatitis, and the present invention provides intermediates in production of the [2-(3,3,5,5-tetramethylcyclohexyl)phenyl]piperazine compounds suitable for industrial large-scale production.

### Means for Solving the Problems

As a result of intensive research, the present inventors have discovered production methods of [2-(3,3,5,5-tetramethylcyclohexyl)phenyl]piperazine compounds and intermediates in the production, which is suitable for industrial large-scale production in the following points: (1) purification by column chromatography is not used, (2) explosive intermediates in production are not used, (3) halogenated solvents are not used, and (4) relatively less expensive materials are used. And the present invention was completed on the basis of the discovery.

Specifically, the invention includes the followings:

[1] A compound represented by the following general formula (A) or salt thereof:

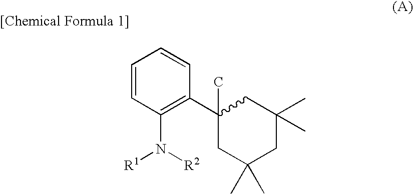

wherein R¹ and R² independently represent hydrogen or a protecting group for an amino group, or R¹ and R² may bond together to form an optionally substituted pyrrole ring, an optionally substituted piperidine ring or an optionally substituted piperazine ring,

the bond (B) represented by the following formula represents a single bond or a double bond

[Chemical Formula 2]

[symbol]

(B)

and C represents hydroxyl or hydrogen when the bond (B) represents a single bond and C is absent when the bond (B) represents a double bond.

[2] The compound or salt thereof according to [1], wherein the compound is represented by the general formula (A-1):

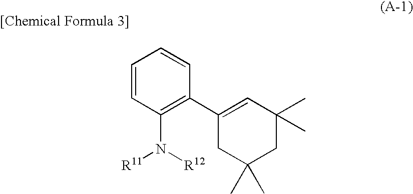

wherein R¹¹ and R¹² independently represent hydrogen or a protecting group for an amino group, or R¹¹ and R¹² may bond together to form an optionally substituted pyrrole ring or an optionally substituted piperidine ring.

[3] The compound or salt thereof according to [1], wherein the compound is represented by the general formula (A-2):

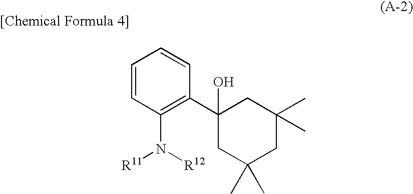

wherein R¹¹ and R¹² independently represent hydrogen or a protecting group for an amino group, or R¹¹ and R¹² may bond together to form an optionally substituted pyrrole ring or an optionally substituted piperidine ring.

[4] The compound or salt thereof according to [1], wherein the compound is represented by the formula (A-3):

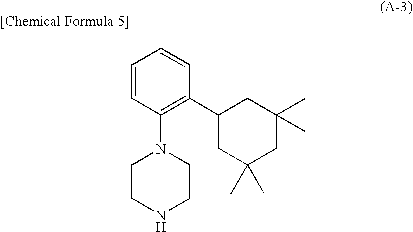

[5] The compound or salt thereof according to any of [1] to [3], wherein the protecting group for an amino group is formyl, pivaloyl, trifluoroacetyl, trichloroacetyl, acetyl, phenylacetyl, benzoyl, N,N-dimethylaminocarbonyl, N-[2-trimethylsilylethoxy]methyl, t-butyloxycarbonyl, methyloxycarbonyl, ethyloxycarbonyl, 2,2,2-trichloroethyloxycarbonyl, 2-trimethylsilyloxycarbonyl, vinyloxycarbonyl, allyloxycarbonyl, benzyloxycarbonyl, p-methoxybenzyloxycarbonyl, p-nitrobenzyloxycarbonyl or benzyl.
[6] The compound or salt thereof according to [1] or [5], wherein R² is hydrogen.
[7] The compound or salt thereof according to [1], wherein R² is hydrogen and R¹ is hydrogen, formyl or pivaloyl.
[8] The compound or salt thereof according to any of [2], [3] and [5], wherein R¹² is hydrogen.
[9] The compound or salt thereof according to [2] or [3], wherein R¹² is hydrogen and R¹¹ is hydrogen, formyl or pivaloyl.
[10] A salt of the compound according to any of [1] to [4] with an acid selected from oxalic acid, fumaric acid, p-toluenesulfonic acid or methanesulfonic acid.
[11] A salt of the compound according to any of [1] to [4] with methanesulfonic acid.

### EFFECT OF THE INVENTION

The present invention can provide useful production methods of [2-(3,3,5,5-tetramethylcyclohexyl)phenyl]piperazine compounds having cell adhesion inhibitory action and cell infiltration inhibitory action as well as compounds or salts thereof useful as intermediates in the production.

### BEST MODE FOR CARRYING OUT THE INVENTION

The present invention will now be explained in detail.

Throughout the present specification, the structural formulas for the compounds will show only one specific isomer for convenience, but the invention includes all isomers such as geometric isomers, optical isomers, stereoisomers and tautomers implied by the compound structures, as well as their isomer mixtures, and the compounds may therefore be any of the isomers or their mixtures, without being limited to the formulas shown for convenience. The compounds of the invention may therefore be in optically active or racemic form, both of which are included without restrictions according to the invention. Polymorphic crystals may also exist, and there may be used any crystal form or a mixture thereof without any restrictions, while the compounds of the invention include both anhydrous and hydrated forms.

The definitions of the terms and symbols used throughout the present specification will now be explained, prior to a more detailed description of the invention.

A “halogen” refers to fluorine, chlorine, bromine or iodine.

The term “C1-6 alkyl” refers to a straight-chain or branched C1-6 alkyl group, and as specific examples there may be mentioned methyl, ethyl, 1-propyl (n-propyl), 2-propyl (i-propyl), 2-methyl-1-propyl (1-butyl), 2-methyl-2-propyl (t-butyl), 1-butyl (n-butyl) and 2-butyl (s-butyl).

The term “cyclopropyl-C₁₋₆ alkyl” refers to the above “C₁₋₆ alkyl” bonded to a cyclopropyl group, and as specific examples there may be mentioned cyclopropylmethyl, 2-cyclopropylethyl and 3-cyclopropylpropyl.

The term “C3-8 cycloalkyl” refers to a C3-8 monocyclic saturated aliphatic hydrocarbon group, and as specific examples there may be mentioned cyclopropyl, cyclobutyl, cyclopentyl, cyclohexyl, cycloheptyl and cyclooctyl.

The term “a protecting group for an amino group” is not limited so long as it is a group used for a protecting group for an amino group (Protective Groups in Organic Synthesis, John Wiley & Sons, Inc.), and as specific examples there may be mentioned formyl, pivaloyl, trifluoroacetyl, trichloroacetyl, acetyl, phenylacetyl, benzoyl, N,N-dimethylaminocarbonyl, N-[2-trimethylsilylethoxy]methyl, t-butyloxycarbonyl, methyloxycarbonyl, ethyloxycarbonyl, 2,2,2-trichloroethyloxycarbonyl, 2-trimethylsilyloxycarbonyl, vinyloxycarbonyl, allyloxycarbonyl, benzyloxycarbonyl, p-methoxybenzyloxycarbonyl, p-nitrobenzyloxycarbonyl or benzyl.

[The meanings of R¹ and R²]

R¹ and R² independently represent hydrogen or a protecting group for an amino group, or R¹ and R² may bond together to form an optionally substituted pyrrole ring, an optionally substituted piperidine ring or an optionally substituted piperazine ring.

Preferable examples of R¹ are hydrogen, formyl, pivaloyl, trifluoroacetyl, trichloroacetyl, acetyl, or N,N-dimethylaminocarbonyl, more preferable examples are hydrogen, formyl or pivaloyl, still more preferable examples are formyl or pivaloyl, and the most preferable example is formyl.

Preferable examples of R² are hydrogen, formyl, pivaloyl, trifluoroacetyl, trichloroacetyl, acetyl, or N,N-dimethylaminocarbonyl, more preferable examples are hydrogen, formyl or pivaloyl, still more preferable example is hydrogen.

“Substituent” in “an optionally substituted pyrrole ring” is not limited so long as it is “an optionally substituted pyrrole ring” used as a protecting group for an amino group. Preferable examples of “an optionally substituted pyrrole ring” are a pyrrole ring having 1 to 4 C1-6 alkyl, more preferable examples are a 2,5-dimethylpyrrole ring or a pyrrole ring.

“Substituent” in “an optionally substituted piperidine ring” is not limited so long as it is “an optionally substituted piperidine ring” used as a protecting group for an amino group. Preferable examples of “an optionally substituted piperidine ring” are a non-substituted piperidine ring.

“Substituent” in “an optionally substituted piperazine ring” is not limited so long as it is “an optionally substituted piperazine ring” used as a protecting group for an amino group. Preferable examples of “an optionally substituted piperazine ring” are a non-substituted piperazine ring.

[The meanings of R¹¹ and R¹²]

R¹¹ and R¹² independently represent hydrogen or a protecting group for an amino group, or R¹¹ and R¹² may bond together to form an optionally substituted pyrrole ring or an optionally substituted piperidine ring.

Preferable examples of R¹¹ are hydrogen, formyl, pivaloyl, trifluoroacetyl, trichloroacetyl, acetyl, or N,N-dimethylaminocarbonyl, more preferable examples are hydrogen, formyl or pivaloyl, still more preferable examples are formyl or pivaloyl, and the most preferable example is formyl.

Preferable examples of R¹² are hydrogen, formyl, pivaloyl, trifluoroacetyl, trichloroacetyl, acetyl, or N,N-dimethylaminocarbonyl, more preferable examples are hydrogen, formyl or pivaloyl, still more preferable example is hydrogen.

“Substituent” in “an optionally substituted pyrrole ring” is not limited so long as it is “an optionally substituted pyrrole ring” used as a protecting group for an amino group. Preferable examples of “an optionally substituted pyrrole ring” are a pyrrole ring having 1 to 4 C1-6 alkyl, more preferable examples are a 2,5-dimethylpyrrole ring or a pyrrole ring.

“Substituent” in “an optionally substituted piperidine ring” is not limited so long as it is “an optionally substituted piperidine ring” used as a protecting group for an amino group. Preferable examples of “an optionally substituted piperidine ring” are a non-substituted piperidine ring.

[The meanings of R³]

R³ which is described later represents hydrogen, C1-6 alkyl, cyclopropyl-C1-6 alkyl or a protecting group for an amino group, and preferable examples of R³ are hydrogen, C1-6 alkyl, cyclopropyl-C1-6 alkyl, t-butyloxycarbonyl, benzyloxycarbonyl, formyl, pivaloyl, trifluoroacetyl, trichloroacetyl, acetyl, phenylacetyl, benzoyl, p-methoxybenzyl or benzyl, more preferable examples are hydrogen, t-butyloxycarbonyl or benzyloxycarbonyl, still more preferable examples are hydrogen or t-butyloxycarbonyl, and the most preferable example is hydrogen.

[The meanings of R⁶]

R⁶ which is described later represents C₁₋₆ alkyl or C₃₋₆ cycloalkyl, and preferable examples of R⁶ are n-propyl or cyclopropyl, and more preferable example is cyclopropyl.

[The meanings of X¹ and X²]

X¹ and X² which are described later independently represent a leaving group, and preferable examples of X¹ and X² are halogen, methanesulfonyloxy or p-toluenesulfonyloxy, more preferable examples are chlorine, bromine, iodine, methanesulfonyloxy or p-toluenesulfonyloxy, still more preferable examples are chlorine or bromine, and the most preferable example is chlorine.

A “salt” as referred to throughout the present specification is not particularly limited so long as it is formed with the compound of the invention, and as examples there may be mentioned inorganic acid salts, organic acid salts and acidic amino acid salts.

As preferable examples of inorganic acid salts there may be mentioned hydrochloride, hydrobromide, sulfate, nitrate and phosphate, and as preferable examples of organic acid salts there may be mentioned oxalate, acetate, succinate, fumarate, maleate, tartarate, citrate, lactate, stearate, benzoate, methanesulfonate, ethanesulfonate, p-toluenesulfonate and benzenesulfonate.

As preferable examples of acidic amino acid salts there may be mentioned aspartate and glutamate.

An acid may form a salt in an appropriate ratio of 0.1 to 5 molecules with 1 molecule of the compound, and preferably forms a salt in an appropriate ration of approximately 1 molecule with 1 molecule of the compound.

The production methods according to the present invention will be explained in detail.

In each formula below, R¹, R², R⁶, X¹ and X² have the same definitions as R¹, R², R⁶, X¹ and X² above, respectively, and T¹ represents leaving group (for example, halogen, methanesulfonyloxy or p-toluenesulfonyloxy, and preferably chlorine).

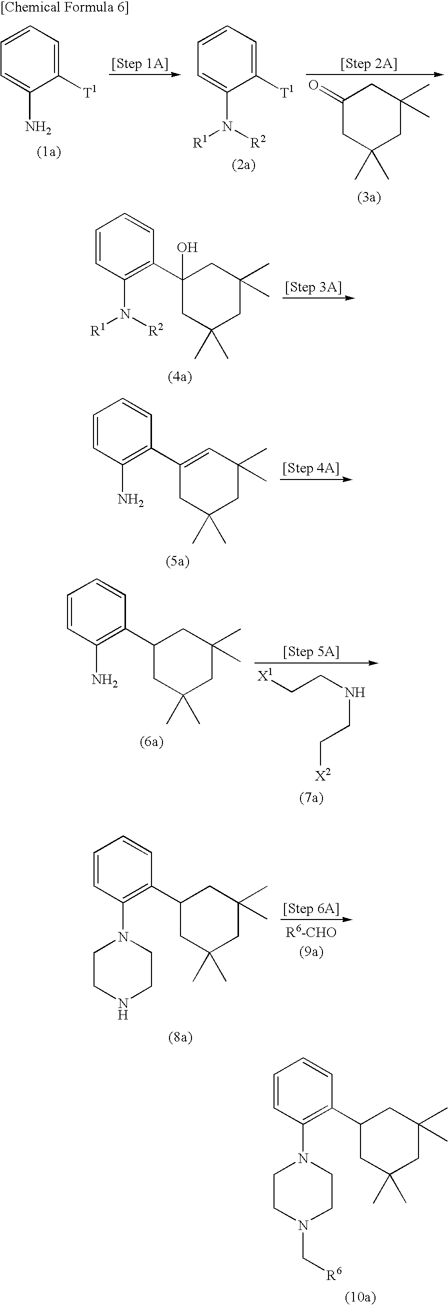

[Step 1A]

This step is a step of producing compound (2a) by the reaction of compound (1a) with a reagent of introducing a protecting group in a solvent. In this step, the reaction can be carried out in the presence of a base or acid anhydride.

This step can be carried out according to the generally used methods such as those described in Protective Groups in Organic Synthesis, John Wiley & Sons, Inc. and Tetrahedron 1983, 39, 3767-3776. This step can be carried out under the stream or atmosphere of inert gas such as nitrogen or argon.

As compound (1a) may be used publicly known compounds, commercially available compounds or compound easily prepared from commercially available compounds by the methods usually done by those skilled in the art.

The solvent used for this step is not particularly limited so long as it dissolves starting materials to some extent and does not inhibit the reaction, and as examples there may be mentioned ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are tetrahydrofuran or 1,2-dimethoxyethane.

The reagent of introducing a protecting group is not particularly limited so long as it can introduce a protecting group to nitrogen, and as examples there may be mentioned a reagent for introducing formyl, pivaloyl, trifluoroacetyl, trichloroacetyl, acetyl, phenylacetyl, benzoyl, N,N-dimethylaminocarbonyl, N-[2-trimethylsilylethoxy]methyl, t-butyloxycarbonyl, methyloxycarbonyl, ethyloxycarbonyl, 2,2,2-trichloroethyloxycarbonyl, 2-trimethylsilyloxycarbonyl, vinyloxycarbonyl, allyloxycarbonyl, benzyloxycarbonyl, p-methoxybenzyloxycarbonyl, p-nitrobenzyloxycarbonyl or benzyl, and preferable examples are an acylating reagent or a formylating reagent.

The above acylating reagent refers to pivaloyl chloride, trifluoroacetyl chloride, acetyl chloride, trichloroacetyl chloride, etc., and preferably pivaloyl chloride.

The above formylating reagent refers to combination of acid anhydride such as acetic anhydride and formic acid, or phenyl formate, etc., and preferably combination of acetic anhydride and formic acid.

The above base refers to triethylamine, diisopropylethylamine, pyridine, potassium carbonate, etc., preferably triethylamine.

The above acid anhydride refers to acetic anhydride, propionic anhydride, etc., and preferably acetic anhydride.

The reaction temperature will generally differ depending on the starting materials, the solvent, the other reagents used in the reaction, and it is preferably 0° C. to 100° C. (internal temperature of the reaction vessel) and more preferably 20° C. to 50° C. (internal temperature of the reaction vessel). The reaction time will generally differ depending on the starting materials, the solvent, the other reagents used in the reaction, and the reaction temperature, and stirring at the above reaction temperature for 1 to 5 hours after the addition of the reagents is preferable and stirring for approximately 3 hours is more preferable.

The acylating reagent can be used in an amount of 1- to 2-fold molar amount with respect to compound (1a), and preferably it is used in an amount of 1.05- to 1.25-fold molar.

The formylating reagent can be used in an amount of 1- to 10-fold molar amount with respect to compound (1a), and preferably it is used in an amount of 1.1- to 4-fold molar.

The above base can be used in an amount of 1- to 10-fold molar with respect to compound (1a), and preferably it is used in an amount of 1- to 3-fold molar.

The above acid anhydride can be used in an amount of 1- to 10-fold molar with respect to compound (1a), and preferably it is used in an amount of 2- to 4-fold molar.

[Step 2A]

This is a step of producing compound (4a) by the reaction of anionized compound (2a), obtainable by the reaction of compound (2a) with a base, with compound (3a) in a solvent. As compound (3a) may be used commercially available compounds.

This step can be carried out according to the generally used methods such as those described in J. Chem. Soc. Perkin. Trans. 1, 1986, 349-359, J. Org. Chem., 1984, 49, 2063-2065, Tetrahedron, 1992, 48, 167-176, and Tetrahedron, 1983, 39, 3767-3776.

This step can be carried out under the stream or atmosphere of inert gas such as nitrogen or argon.

The solvent used for this step is not particularly limited so long as it dissolves starting materials to some extent and does not inhibit the reaction, and as examples there may be mentioned ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are tetrahydrofuran or 1,2-dimethoxyethane.

The above base refers to a butyllithium reagent alone such as n-butyllithium, s-butyllithium and t-butyllithium, or combination of a base selected from Base Group B with the above butyllithium reagent, wherein Base Group B consists of sodium hydride, methyllithium, phenyllithium, potassium hydride, lithium methoxide, sodium methoxide, sodium ethoxide and potassium t-butoxide. Preferable butyllithium reagents include n-butyllithium or t-butyllithium.

Base Group B preferably consists of sodium hydride, methyllithium and phenyllithium, and more preferably consists of sodium hydride and methyllithium. The reaction temperature will generally differ depending on the starting materials, the solvent, the other reagents used in the reaction, and it is preferably—100° C. to 50° C. (internal temperature of the reaction vessel) and more preferably—80° C. to 0° C. (internal temperature of the reaction vessel).

The reaction time will generally differ depending on the starting materials, the solvent, the other reagents used in the reaction, and the reaction temperature. Preferably, compound (2a) and a base are stirred at the above reaction temperature for 1 to 3 hours to produce anionized compound (2a). Then, compound (3a) is added to the reaction mixture, followed by stirring at the above temperature for 1 to 20 hours to produce compound (4a). In this case, anionized compound (2a) can be added to the solution containing compound (3a).

More preferably, compound (2a) and a base are stirred at the above reaction temperature for 0.5 to 1 hour, and compound (3a) is added to the reaction mixture, followed by stirring at the above temperature for 1 to 3 hours to produce compound (4a).

The above base can be used in an amount of 1- to 3-fold molar with respect to compound (2a), and preferably it is used in an amount of 1- to 2.2-fold molar.

Compound (3a) can be used in an amount of 0.3- to 3-fold molar with respect to compound (2a), and preferably it is used in an amount of 0.4- to 1-fold molar, and more preferably in an amount of 0.6- to 0.8-fold molar.

[Step 3A]

This is a step of producing compound (5a) by the reaction of compound (4a) in a solvent in the presence of an acid. This step can be carried out according to the methods generally used for dehydration reaction of alcohol compounds.

This step can be carried out under the stream or atmosphere of inert gas such as nitrogen or argon.

The solvent used for this step is not particularly limited so long as it dissolves starting materials to some extent and does not inhibit the reaction, and as examples there may be mentioned alcohol solvents such as methanol, ethanol, propanol and butanol, ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are methanol, toluene or heptane.

The above acid refers to pyridinium p-toluenesulfonate, p-toluenesulfonic acid, hydrochloric acid, sulfuric acid, etc., preferably pyridinium p-toluenesulfonate.

The reaction temperature will generally differ depending on the starting materials, the solvent, the other reagents used in the reaction, and it is preferably 30° C. to 150° C. (internal temperature of the reaction vessel) and more preferably 70° C. to 120° C. (internal temperature of the reaction vessel).

The reaction time will generally differ depending on the starting materials, the solvent, the other reagents used in the reaction, and the reaction temperature, and stirring at the above reaction temperature for 0.5 to 5 hours after the addition of the reagents is preferable and stirring for approximately 1 hour is more preferable.

The above acid can be used in an amount of 0.01- to 10-fold molar with respect to compound (4a), and preferably it is used in an amount of 0.05- to 1-fold molar and more preferably in an amount of 0.08- to 0.2-fold molar.

[Step 3A(2)]

In step 3A, a mixture of compound (5a-2) represented by the formula:

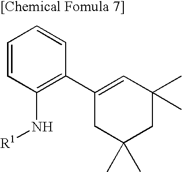

(5a-2)

wherein R¹ has the same meaning as R¹ above, and compound (5a) may be obtained. In this case, compound (5a) can be obtained by allowing the mixture of compound (5a-2) and compound (5a) to further react in the presence of a base.

This step can be carried out according to the generally used methods such as those described in Tetrahedron Letters 2004, 45, 9405-9407 and J. Am. Chem. Soc., 1991, 113, 5085-5086.

This step can be carried out under the stream or atmosphere of inert gas such as nitrogen or argon.

The above base refers to sodium hydroxide, potassium hydroxide, lithium hydroxide, potassium carbonate, sodium carbonate, lithium carbonate, sodium methoxide, potassium methoxide, methylamine, ethylamine, ammonia, etc., and preferably sodium hydroxide or potassium hydroxide.

The solvent used for this step is not particularly limited so long as it dissolves starting materials to some extent and does not inhibit the reaction, and as examples there may be mentioned alcohol solvents such as methanol, ethanol, propanol and butanol, ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are methanol or ethanol. The reaction temperature will generally differ depending on the starting materials, the solvent, the other reagents used in the reaction, and it is preferably 20° C. to 240° C. (internal temperature of the reaction vessel) and more preferably 60° C. to 180° C. (internal temperature of the reaction vessel).

The reaction time will generally differ depending on the starting materials, the solvent, the other reagents used in the reaction, and the reaction temperature, and stirring at the above reaction temperature for 1 to 30 hours after the addition of the reagents is preferable and stirring for approximately 15 hours is more preferable.

The base can be used in an amount of 0.1- to 100-fold molar with respect to total amount of compound (5a-2) and compound (5a), and preferably it is used in an amount of 0.5- to 2-fold molar.

[Step 4A]

This is a step of producing compound (6a) by the reaction of compound (5a) in a solvent in the presence of a reduction catalyst under hydrogen atmosphere.

This step can be carried out according to the generally used methods such as those described in Jikken Kagaku Koza, 4th edition (4th series of experimental chemistry), vol. 26, p 251-266.

The solvent used for this step is not particularly limited so long as it dissolves starting materials to some extent and does not inhibit the reaction, and as examples there may be mentioned alcohol solvents such as methanol, ethanol, propanol and butanol, ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are methanol or ethanol.

The above reduction catalyst refers to palladium on carbon, palladium hydroxide, platinum oxide, Raney nickel, and preferably palladium on carbon.

In this step, the reaction can be carried out under hydrogen atmosphere at atmospheric pressure to under pressure (1 to 100 kgf/cm²), and preferably the reaction can be carried out under hydrogen atmosphere at 5 to 15 kgf/cm².

The reaction temperature will generally differ depending on the starting materials, the solvent, the other reagents used in the reaction, and it is preferably 0° C. to 60° C. (internal temperature of the reaction vessel) and more preferably 15° C. to 30° C. (internal temperature of the reaction vessel).

The reaction time will generally differ depending on the starting materials, the solvent, the other reagents used in the reaction, and the reaction temperature, and stirring at the above reaction temperature for 5 to 100 hours after the addition of the reagents is preferable and stirring for approximately 15 hours is more preferable.

The above reduction catalyst can be used in an amount of 0.001- to 1-fold molar with respect to compound (5a), and preferably it is used in an amount of 0.01- to 0.06-fold molar and more preferably in an amount of 0.03-fold molar.

[Step 5A]

This is a step of producing compound (8a) by the reaction of compound (6a) and compound (7a) in a solvent. In this step, the reaction can be carried out in the presence of a base.

This step can be carried out according to the generally used methods such as those described in Chem. Lett., 1998, 8, 2675-2680.

This step can be carried out under the stream or atmosphere of inert gas such as nitrogen or argon. The reaction can be accelerated by combination with microwave.

As compound (7a) may be used publicly know compounds, commercially available compounds or compound easily prepared from commercially available compounds by the methods usually done by those skilled in the art.

The solvent used for this step is not particularly limited so long as it dissolves starting materials to some extent and does not inhibit the reaction, and as examples there may be mentioned aromatic hydrocarbon solvents such as p-cymene, o-dichlorobenzene, diphenyl ether, benzene, toluene and xylene, alcohol solvents such as butanol and ethylene glycol, aliphatic hydrocarbon solvents such as heptane and hexane, ether solvents such as tetrahydrofuran, 1,2-dimethoxyethane, t-butyl methyl ether, cyclopentyl methyl ether, diethyl ether, diisopropyl ether, dibutyl ether and dicyclopentyl ether or mixed solvents thereof, and preferable examples are p-cymene or diphenyl ether.

The above base refers to potassium carbonate, sodium carbonate, triethylamine, etc., and preferably potassium carbonate or sodium carbonate, more preferably potassium carbonate.

The reaction temperature will generally differ depending on the starting materials, the solvent, the other reagents used in the reaction, and it is preferably 50° C. to 250° C. (internal temperature of the reaction vessel) and more preferably 90° C. to 190° C. (internal temperature of the reaction vessel).

The reaction time will generally differ depending on the starting materials, the solvent, the other reagents used in the reaction, and the reaction temperature, and stirring at the above reaction temperature for 2 to 15 hours after the addition of the reagents is preferable and stirring for approximately 8 hours is more preferable.

Compound (7a) can be used in an amount of 1- to 3-fold molar with respect to compound (6a), and preferably it is used in an amount of 1- to 2-fold molar, more preferably 1.2- to 2-fold molar.

The above base can be used in an amount of 1- to 10-fold molar with respect to compound (6a), and preferably it is used in an amount of 2- to 5-fold molar, more preferably 3-fold molar.

In step 5A, compound (7a) in situ synthesized by the reaction of diethanolamine with a halogenating reagent or a reagent for introducing a leaving group in a reaction vessel can be used instead of compound (7a) (Synthetic communication, 1998, 28, 1175-1178).

The halogenating reagent refers to thionyl chloride, hydrochloric acid, phosphorus oxychloride, hydrobromic acid, N-bromosuccinimide, N-chlorosuccinimide, etc., and preferably thionyl chloride or hydrochloric acid.

The reagent for introducing a leaving group refers to mesyl chloride, tosyl chloride, etc.

The reaction of diethanolamine with the halogenating reagent or the reagent for introducing a leaving group can be carried out in the presence of a base such as triethylamine. The reaction can be accelerated by combination with microwave.

[Step 6A]

This is a step of producing compound (10a) by the reaction of compound (8a) and compound(9a) in a solvent in the presence of a reducing agent.

This step can be carried out according to the generally used methods such as those described in Jikken Kagaku Koza, 4th edition (4th series of experimental chemistry), vol. 20, p 282-284.

This step can be carried out under stream or atmosphere of inert gas such as nitrogen or argon.

As compound (9a) may be used publicly know compounds, commercially available compounds or compound easily prepared from commercially available compounds by the methods usually done by those skilled in the art.

The solvent used for this step is not particularly limited so long as it dissolves starting materials to some extent and does not inhibit the reaction, and as examples there may be mentioned ether solvents such as tetrahydrofuran, 1,2-dimethoxyethane, t-butyl methyl ether, cyclopentyl methyl ether, diethyl ether, diisopropyl ether, dibutyl ether and dicyclopentyl ether, aromatic hydrocarbon solvents such as benzene and toluene, aliphatic hydrocarbon solvents such as heptane and hexane, and mixed solvents thereof, and preferable examples are tetrahydrofuran or toluene.

The above reducing reagent refers to sodium triacetoxyborohydride, sodium cyanoborohydride, sodium borohydride, etc., and preferably sodium triacetoxyborohydride.

The reaction temperature will generally differ depending on the starting materials, the solvent, the other reagents used in the reaction, and it is preferably 0° C. to 50° C. (internal temperature of the reaction vessel) and more preferably 10° C. to 30° C. (internal temperature of the reaction vessel).

The reaction time will generally differ depending on the starting materials, the solvent, the other reagents used in the reaction, and the reaction temperature, and stirring at the above reaction temperature for 1 to 5 hours after the addition of the reagents is preferable and stirring for approximately 1 hour is more preferable.

Compound (9a) can be used in an amount of 1- to 2-fold molar with respect to compound (8a), and preferably it is used in an amount of 1- to 1.5-fold molar and more preferably in an amount of 1- to 1.2-fold molar.

The above reducing reagent can be used in an amount of 0.3- to 2-fold molar with respect to compound (8a), and preferably it is used in an amount of 1.0- to 2.0-fold molar and more preferably in an amount of 1.3- to 1.5-fold molar.

[Step 7A]

This is a step of producing compound (12a) by the reaction of compound (6a) and compound (11a) in a solvent.

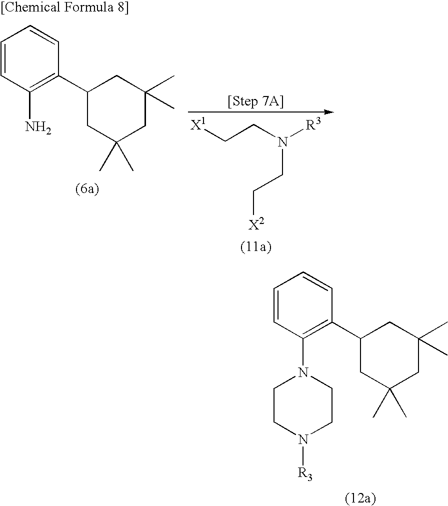

In each formula, R³, X¹ and X² have the same definitions as R³, X¹ and X² above, respectively.

This step can be carried out according to the same methods and conditions as the above step 5A.

As compound (11a) may be used publicly know compounds, commercially available compounds or compound easily prepared from commercially available compounds by the methods usually done by those skilled in the art.

In step 7A, compound (11a) obtainable by the reaction of compound (11a-2) represented by the formula

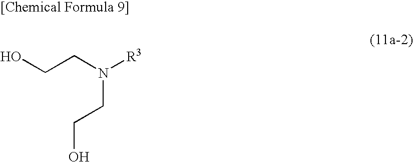

wherein R³ has the same definition as R³ above, with a halogenation reagent or a reagent for introducing a leaving group in a reaction vessel can be used instead of compound (11a). This step can be carried out according to the same methods and conditions as the above step 5A.

The conversion from compound (4a) to compound (6a) can be carried out also by the [Step 3B] to [Step 5B] below.

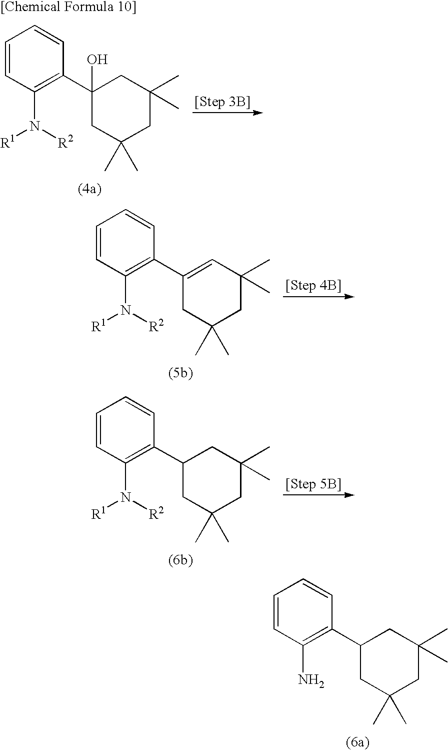

In each formula, R¹ and R² have the same definitions as R¹ and R² above, respectively.

[Step 3B]

This is a step of producing compound (5b) of the present invention by the reaction of compound (4a) in a solvent in the presence of an acid.

This step can be carried out according to the same methods and conditions as the above step 3A.

[Step 4B]

This is a step of producing compound (6b) by the reaction of compound (5b) in a solvent in the presence of a reduction catalyst under hydrogen atmosphere.

This step can be carried out according to the same methods and conditions as the above step 4A.

[Step 5B]

This is a step of producing compound (6a) by the reaction of compound (6b) in the presence or absence of a solvent, in a base.

This step can be carried out according to the same methods and conditions as the above step 3A(2).

After completion of the reactions of each of the methods and steps described above, the target compound of each step may be recovered from the reaction mixture according to conventional procedures.

For example, when the entire reaction mixture is a liquid, it may be returned to room temperature or cooled on ice if necessary, then allowed to neutralize an acid, an alkali, an oxidizing agent or a reducing agent if necessary, and then water and an organic solvent such as ethyl acetate which is immiscible with water and which does not react with the target compound may be added, and the layer containing the target compound is separated. Next, there may be added a solvent which is immiscible with the resultant layer and which does not react with the target compound, and the layer containing the target compound may be washed and separated. If the layer is an organic layer, it may be dried using a desiccant such as anhydrous magnesium sulfate or anhydrous sodium sulfate, the solvent may be distilled off to recover the target compound. If the layer is an aqueous layer, it may be electrically desalted and then lyophilized to recover the target compound.

When the entire reaction mixture is a liquid, if possible the substances other than the target compound (for example, solvents, reagents, etc.) may be simply distilled off at atmospheric pressure or under reduced pressure to recover the target compound.

When the target compound alone precipitates as a solid, or when the entire reaction mixture is a liquid and the target compound alone precipitates as a solid during the recovery procedure, the target compound may be first filtered by a filtration method and the filtered target compound washed with a suitable organic or inorganic solvent and dried to allow treatment of the mother liquor in the same manner as when the entire reaction mixture is a liquid, in order to recover the target compound.

When only the reagent or catalyst is present in solid form, or when the entire reaction mixture is a liquid and the reagent or catalyst alone precipitates as a solid during the recovery procedure, with the target compound dissolved in the solution, the reagent or catalyst may be first filtered by a filtration method and the filtered reagent or catalyst washed with a suitable organic or inorganic solvent, and then the obtained wash liquids combined with the mother liquor and the obtained mixture treated in the same manner as when the entire reaction mixture is a liquid, in order to recover the target compound.

Particularly when substances other than the target compound in the reaction mixture do not inhibit the reaction of the subsequent step, the reaction mixture may be used directly for the subsequent step without isolation of the target compound.

The purity of the target compound recovered by the method described above may be improved by appropriately employing a recrystallization method, chromatography method or distillation method.

When the recovered target compound is a solid, it will usually be possible to improve the purity of the target compound by recrystallization. For recrystallization, a single solvent or multiple solvents which do not react with the target compound may be used. Specifically, the target compound is first dissolved in the single or multiple solvents which do not react therewith, either at room temperature or with heating. The resulting solution is either cooled on ice or allowed to stand at room temperature to crystallization of the target compound from the mixture.

When the target compound is free form, it can form a salt with an acid or base, and the salt of the target compound can be purified by recrystallization same as described above. After purification, the salt of the target compound can be converted to a free form to produce the target compound with higher purity.

When the recovered target compound is a liquid, the purity of the target compound may be improved by any of various chromatography methods. A weak acid silica gel such as Silica Gel 60 (70-230 mesh or 340-400 mesh) by Merck Co. or BW-300 (300 mesh) by Fuji Silysia Chemical Ltd. may be used in most cases.

When the target compound is basic and adsorption is too strong on the aforementioned silica gels, Chromatorex-NH silica gel (200-350 mesh) propylamine coated one by Fuji Silysia Chemical Ltd. or the like may be used.

When the target compound is dipolar or must be eluted with a polar solvent such as methanol, NAM-200H or NAM-300H by Nam Research Co. may be used. These silica gels may be used for elution of the target compound with a single solvent or multiple solvents which do not react with the target compound, followed by distilling off of the solvent, to yield the target compound with improved purity.

When the recovered target compound is a liquid, its purity may be improved by a distillation method. For distillation, the target compound is subjected to reduced pressure at room temperature or with heating to distill off the target compound.

Representative examples of production methods of the invention have been described above, but the starting compounds and reagents used for production of the compounds of the invention may also form salts, hydrates or solvates, which will differ depending on the starting materials and solvents used, and are not particularly limited so long as they do not inhibit the reaction. The solvents used will also differ depending on the starting materials and reagents, but of course they are not particularly limited so long as they dissolve the starting materials to some extent and do not inhibit the reaction.

When an intermediate in production of the invention are obtained in the free form, a conventional procedure may be carried out to convert it to a salt or hydrate of the intermediate in production.

When an intermediate in production of the invention is obtained as a salt or hydrate, it may be converted to the free form according to a conventional procedure.

The production methods or intermediates in production of the invention may be performed or produced by the methods described in the following examples. However, these specific examples are merely illustrative and are not intended to restrict the invention in any way, and various modifications may be implemented such as are within the scope of the invention.

The compounds accompanying literatures were prepared according to the literatures.

The term “room temperature” in the Reference Examples and the Examples below ordinarily refers to a temperature between approximately 10° C. and 35° C. The percentage values are weight percentages, unless otherwise specified. The other symbols as used herein stand for the followings.

s: singlet
d: doublet
t: triplet
q: quartet
m: multiplet
br: broad
J: coupling constant

### Hz: Hertz

CDCl₃: deutero chloroform
NMR: proton nuclear magnetic resonance
Piv: a pivaloyl group (a t-butylcarbonyl group)

### EXAMPLES

Silica gel used in the following Examples is silica gel 60 (Merck & Co., Inc) or BW300 (Fuji Silysia Chemical Ltd.) unless otherwise mentioned, and NH silica gel used is Chromatorex-NH silica gel (Fuji Silysia Chemical Ltd.), propylamine-coated one.

### Example 1

### 1-Cyclopropylmethyl-4-[2-(3,3,5,5-tetramethylcyclohexyl)phenyl]piperazine methanesulfonate

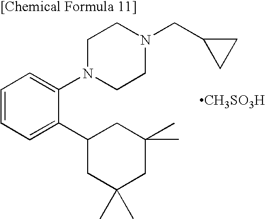

### Example 1-A

### N-(2-Bromophenyl)formamide

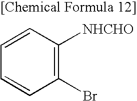

To acetic anhydride (74.2 g, 727 mmol) was added formic acid (32.9 mL, 872 mmol) under a nitrogen atmosphere while stirring at room temperature, followed by stirring at 70° C. for 3 hours. The reaction mixture was allowed to cool down to room temperature, and tetrahydrofuran (50 mL) was added. To the solution was added a solution of 2-bromoaniline (50.0 g, 291 mmol) in tetrahydrofuran (50 mL) at room temperature, followed by stirring at the same temperature for 1 hour, and concentration was carried out. To the resultant crude crystals was added ethanol (200 mL), followed by heating and stirring at 60° C. After the crystals dissolved, the mixture was allowed to cool down to room temperature, then water (400 mL) was added, followed by stirring for 3 hours. The precipitated crystals were collected by filtration to give 48.8 g of the title compound as white crystals.

¹H NMR (400 MHz, CDCl₃) δ: 7.01 (1H, m), 7.22-37 (1.7H, m), 7.50-7.60 (0.6H, m), 7.60 (0.3H, d, J=8 Hz, NH), 7.64 (0.7H, brs, NH), 8.39 (0.7H, dd, J=1 Hz, 8 Hz), 8.49 (0.7H, s), 8.70 (0.3H, d, J=11 Hz).

### Example 1-B

### N-[2-(1-Hydroxy-3,3,5,5-tetramethylcyclohexyl)phenyl]formamide

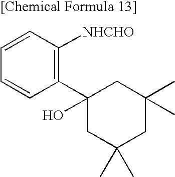

To a solution of sodium hydride (content 60%, 360 mg, 9.00 mmol) in tetrahydrofuran (5 mL) was added dropwise a solution of N-(2-bromophenyl)formamide (1.50 g, 7.50 mmol) in tetrahydrofuran (5 mL) while stirring at room temperature under a nitrogen atmosphere, followed by stirring at the same temperature for 30 minutes. The reaction mixture was cooled to −78° C., n-butyllithium (2.67 M solution in hexane, 3.37 mL, 9.00 mmol) was added dropwise thereto, followed by stirring at the same temperature for 30 minutes. To the reaction mixture was further added dropwise a solution of 3,3,5,5-tetramethylcyclohexanone (771 mg, 5.00 mmol) in tetrahydrofuran (1 mL) at the same temperature, followed by stirring at the same temperature for 1 hour and at room temperature for 1 hour. To the reaction mixture were added water (10 mL), thereafter extraction with ethyl acetate was carried out. The organic layer was washed with water and a 5% aqueous solution of sodium chloride, dried over anhydrous magnesium sulfate, filtrated, and concentrated. To the obtained crude crystals was added ethanol (7.7 mL), followed by heating and stirring at 60° C., and after the crystals were dissolved, the mixture was allowed to cool down to room temperature. Upon confirming the precipitation of crystals, water (6 mL) was added to the mixture, followed by stirring for 2 hours. The crystals were collected by filtration and dried to give 974 mg of the title compound as yellow crystals.

¹H NMR (400 MHz, CDCl₃) δ: 0.95 (6H, s), 1.17 (1H, m), 1.26 (1H, s), 1.32 (6H, s), 1.50 (1H, m), 1.60 (2H, m), 2.00 (2H, m), 7.00-7.33 (3.4H, m), 8.30 (0.6H, d, J=8 Hz), 8.44 (0.6H, s), 8.63 (0.4H, d, J=12 Hz), 9.72 (0.4H, brd, J=9 Hz, NH), 10.10 (0.6H, brs, NH).

### Example 1-C

### 2-(3,3,5,5-Tetramethylcyclohex-1-enyl)phenylamine

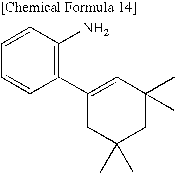

To a solution of N-[2-(1-hydroxy-3,3,5,5-tetramethylcyclohexyl)phenyl]formamide (4.00 g, 14.5 mmol) in toluene (40 mL) was added pyridinium p-toluenesulfonate (365 mg, 1.45 mmol) at room temperature, followed by stirring at 110° C. for 1 hour. Then, the reaction mixture was cooled down to 50° C., methanol (40 mL) and a 5N aqueous solution of sodium hydroxide (14.5 mL) were added thereto, and the reaction mixture was stirred at 80° C. for 14 hours. The reaction mixture was allowed to cool down to room temperature, the aqueous layer was removed. The organic layer was washed with water and a 5% aqueous solution of sodium chloride, dried over anhydrous magnesium sulfate, filtered, and concentrated to give 3.33 g of the title compound as a brown oil.

¹H NMR (400 MHz, CDCl₃) δ: 1.05 (6H, s), 1.09 (6H, s), 1.42 (2H, s), 2.01 (2H, s), 3.74 (2H, brs, NH₂), 5.51 (1H, s), 6.68 (1H, dd, J=1 Hz, 8 Hz), 6.73 (1H, dt, J=1 Hz, 8 Hz), 6.95 (1H, dd, J=1 Hz, 8 Hz), 7.03 (1H, dt, J=1 Hz, 8 Hz).

### Example 1-D

### 2-(3,3,5,5-Tetramethylcyclohexyl)phenylamine

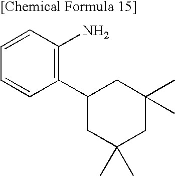

In a 100 mL autoclave, 10% palladium on carbon (water content 50%, 1.2 g) was added to a solution of 2-(3,3,5,5-tetramethylcyclohex-1-enyl)phenylamine (4.00 g, 17.4 mmol) in ethanol (40 mL), followed by stirring at room temperature, kgf/cm² of hydrogen pressure for 5.5 hours. Then the reaction mixture was stirred at room temperature, 10 kgf/cm² of hydrogen pressure for 3 hours, introduction of hydrogen was stopped, and the reaction mixture was stirred at room temperature for 15 hours. The reaction mixture was stirred at room temperature, 10 kgf/cm² of hydrogen pressure for 9.5 hours, introduction of hydrogen was stopped, and the reaction mixture was stirred at room temperature for 13 hours. The reaction mixture was stirred at room temperature, 10 kgf/cm² of hydrogen pressure for 7.5 hours, and the reaction mixture was stirred at room temperature. The pressure was brought to atmospheric pressure, and the reaction mixture was filtered through Celite, and concentrated to give 3.90 g of the title compound as a brown oil.

¹H NMR (400 MHz, CDCl₃) δ: 0.95 (6H, s), 1.13 (6H, s), 1.14-1.38 (4H, m), 1.60 (2H, m), 2.86 (1H, m), 3.62 (2H, brs, NH₂), 6.68 (1H, dd, J=1 Hz, 8 Hz), 6.78 (1H, dt, J=1 Hz, 8 Hz), 7.02 (1H, dd, J=1 Hz, 8 Hz), 7.12 (1H, dt, J=1 Hz, 8 Hz).

### Example 1-E

### 2-(3,3,5,5-Tetramethylcyclohexyl)phenylamine oxalate

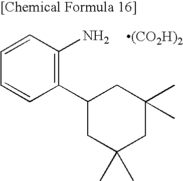

To a solution of 2-(3,3,5,5-tetramethylcyclohexyl)phenylamine (3.90 g, 16.9 mmol) in heptane (19.5 mL) was added dropwise to a solution of oxalic acid (1.82 g, 20.2 mmol) in ethyl acetate (39 mL) while stirring at room temperature, followed by stirring at the same temperature for 66 hours. Precipitated crystals were collected by filtration with glass filter, and dried to give 4.13 g of the title compound as white crystals.

¹H NMR (400 MHz, DMSO-d₆) δ: 0.89 (6H, s), 1.10 (6H, s), 1.11 (3H, m), 1.26 (1H, m), 1.46 (2H, m), 2.87 (1H, m), 3.30 (2H, brs, NH₂), 6.55 (1H, t, J=8 Hz), 6.65 (1H, d, J=8 Hz), 6.87 (1H, t, J=8 Hz), 6.96 (1H, d, J=8 Hz).

### Example 1-F

### 1-[2-(3,3,5,5-Tetramethylcyclohexyl)phenyl]piperazine methanesulfonate

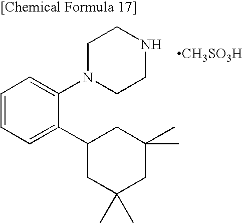

2-(3,3,5,5-Tetramethylcyclohexyl)phenylamine oxalate (4.52 g, 14.1 mmol) was suspended in t-butyl methyl ether (45 mL), and a 1N aqueous solution of potassium hydroxide (16.9 mL) was added, followed by stirring at room temperature for 50 minutes. To the reaction mixture were added t-butyl methyl ether (15 mL) and water (25 mL), and the reaction mixture was stirred at room temperature, and the organic layer was separated. The organic layer was washed with water (23 mL) four times, the solvent was removed under reduced pressure to give 3.18 g of 2-(3,3,5,5-tetramethylcyclohexyl)phenylamine as a red oil.

To a solution of 2-(3,3,5,5-tetramethylcyclohexyl)phenylamine (2.35 g, 10.2 mmol) in p-cymene (24 mL) was added bis(2-chloroethyl)amine hydrochloride (2.19 g, 12.3 mmol), followed by stirring at an external temperature of 180° C. for 8.5 hours. The reaction mixture was allowed to cool down to room temperature, diluted with addition of p-cymene (4 mL), and divided into three portions. To one portion of the reaction mixture was added a 1N aqueous solution of sodium hydroxide (7.8 mL), and the mixture was stirred at room temperature, and the organic layer was separated. The organic layer was washed with water (8 mL) and a 5% aqueous solution of sodium chloride in this order, and diluted with addition of ethyl acetate (9 mL). To the mixture was added methanesulfonic acid (0.19 mL, 2.93 mmol), followed by stirring at room temperature for 1 hour. Produced precipitate was collected by filtration under reduced pressure, washed with ethyl acetate (8 mL), dried at 40° C. under reduced pressure for 1 hour to give 974 mg of crude crystals of the title compound as a light brown solid.

The crude crystals of the title compound (500 mg, content 90.2%, 1.14 mmol) was suspended in toluene (4.5 mL), followed by heating and stirring at 100° C. to completely dissolve the crystals. To the solution was added heptane (2.3 mL), and heating was stopped and stirring was carried out. After crystals were precipitated, the mixture was stirred at room temperature for 5.5 hours. Produced precipitate was collected by filtration under reduced pressure, washed with a mixed solvent of toluene (2.25 mL) and heptane (2.25 mL), and dried at 40° C. under reduced pressure to give 413 mg of the title compound as a light brown solid.

¹H-NMR (400 MHz, CDCl₃) δ: 0.93 (s, 6H), 1.11 (s, 6H), 1.14-1.42 (m, 6H), 2.85 (s, 3H), 3.17 (brs, 4H), 3.39 (brs, 4H), 3.47 (tt, J=13, 3 Hz, 1H), 7.12-7.18 (m, 3H), 7.25-7.26 (m, 1H).

### Example 1-G

### 1-Cyclopropylmethyl-4-[2-(3,3,5,5-tetramethylcyclohexyl)phenyl]piperazine methanesulfonate

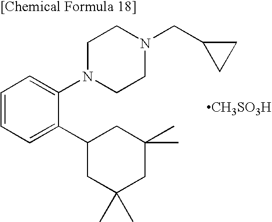

1-[2-(3,3,5,5-Tetramethylcyclohexyl)phenyl]piperazine methanesulfonate (820 mg, 2.07 mmol) was suspended in t-butyl methyl ether (8.2 mL), and a 1N aqueous solution of sodium hydroxide (2.5 mL) was added thereto, followed by stirring at room temperature for 40 minutes. The organic layer was separated and washed twice with water (8 mL), and the solvent was removed under reduced pressure. To the resultant residue were added tetrahydrofuran (6.1 mL), acetic acid (0.116 mL) and cyclopropanecarbaldehyde (0.182 mL) in this order, followed by stirring at room temperature for 20 minutes. To the mixture was added sodium triacetoxyborohydride (606 mg, 2.86 mmol), followed by stirring at room temperature for 1.5 hours. To the reaction mixture were added a 1N aqueous solution of sodium hydroxide (8.1 mL) and t-butyl methyl ether (6.1 mL), followed by stirring at room temperature, and the organic layer was separated. The organic layer was washed twice with water (6 mL), and the solvent was removed under reduced pressure. To the resultant residue was added 4-methyl-2-pentanone (6 mL), followed by heating and stirring at an external temperature of 100° C. Methanesulfonic acid (0.121 mL, 1.86 mmol) was added thereto and dissolved. The mixture was further stirred at an external temperature of 100° C. for 2 minutes, heptane (3 mL) was added, and heating was stopped and stirring was carried out. The mixture was stirred at room temperature for 14 hours and the produced precipitate was collected by filtration under reduced pressure. The precipitate was washed with a mixture of 4-methyl-2-pentanone (3 mL) and heptane (3 mL), and dried under reduced pressure at 40° C. for 2 hours to give 670 mg of the title compound as white crystals.

NMR data for the product corresponded with that for the compound obtained in Example 3-G.

Alternative method for Example 1-B: N-[2-(1-Hydroxy-3,3,5,5-tetramethylcyclohexyl)phenyl]formamide

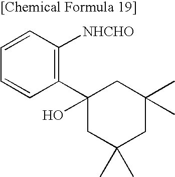

(1) A solution of N-(2-bromophenyl)formamide (1.50 g, 7.50 mmol) in tetrahydrofuran (7.5 mL) was added dropwise to a solution of sodium hydride (content 60%, 360 mg, 9.00 mmol) in tetrahydrofuran (7.5 mL) in a 50 mL three-necked flask under nitrogen atmosphere with stirring at 0° C., followed by stirring at room temperature for 30 minutes.

(2) tetrahydrofuran (3.5 mL) and n-butyllithium (2.67 M solution in hexane, 3.37 mL, 9.00 mmol) were placed in a 100 mL three-necked flask under nitrogen atmosphere, and the solution was cooled to −78° C.

(3) The tetrahydrofuran solution (room temperature) prepared in (1) was added dropwise to the solution prepared in (2) over 2 hours using a dropping funnel. During the addition the reaction mixture was stirred at −78° C. A solution of 3,3,5,5-tetramethylcyclohexanone (771 mg, 5.00 mmol) in tetrahydrofuran (3 mL) was added dropwise to the reaction mixture at −78° C., followed by the stirring at the same temperature for 3 hours. Water (1.5 mL) and tetrahydrofuran (3 mL) were added to the reaction mixture over 30 minutes, and extraction was performed with ethyl acetate. The organic layer was washed with water and a 5% aqueous solution of sodium chloride, dried over anhydrous magnesium sulfate, and filtered to give a solution (31.8 g) containing the title compound. (The content of 881 mg (64%) of the title compound was confirmed by HPLC analysis)

Alternative method for Example 1-C: 2-(3,3,5,5-tetramethylcyclohex-1-enyl)phenylamine

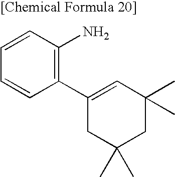

35% hydrochloric acid (265 g) was added to a solution of N-[2-(1-hydroxy-3,3,5,5-tetramethylcyclohexyl)phenyl]formamide (350 g, 1.27 mol) in methanol (1750 mL), followed by stirring at 40° C. for 4 hours. Then the reaction mixture was warmed up to 50° C. and stirred for 1.5 hours, and cooled down to about 25° C.

Toluene (1750 mL) and a 5N aqueous solution of sodium hydroxide (908 g) were added to the reaction mixture. The aqueous layer was discarded, and the organic layer was washed with a 5% aqueous solution of sodium chloride (1750 mL) and water (1750 mL) in this order. The organic layer was filtered to remove insoluble matter, and concentrated under reduced pressure to give the title compound (320 g, 100%) as a pale brown oil.

The product was identical to the compound of Example 1-C by HPLC analysis.

Alternative method for Example 1-D: 2-(3,3,5,5-tetramethylcyclohexyl)phenylamine

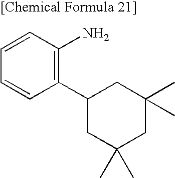

After 5% palladium on carbon (wet 52.7%, 4.51 g), 2-(3,3,5,5-tetramethylcyclohex-1-enyl)phenylamine (15.0 g, 65 mmol) and ethanol (226 mL) was placed in a 500 mL autoclave, and the system was replaced by nitrogen. The system was warmed up to 45° C. and replaced with hydrogen, and the inner pressure was kept at 0.34 MPa.

After stirring for 4 hours with keeping the inner pressure at 0.34 MPa, the system was replaced with nitrogen and cooled down to room temperature.

The solid was removed using a pressurized filter, and the residue was washed with ethanol (75 mL) and concentrated under reduced pressure. Ethyl acetate (75 mL) was added to the resultant oil, which was concentrated under reduced pressure again to give the title compound (14.9 g, 97%) as a pale brown oil.

The product was identical to the compound of Example 1-D by HPLC analysis.

Alternative method for Example 1-E: 2-(3,3,5,5-tetramethylcyclohexyl)phenylamine oxalate

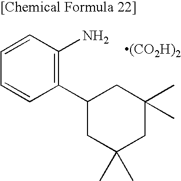

Oxalic acid (121 g) and ethyl acetate (3000 mL) were placed in a 9 L separable flask and the mixture was heated to about 45° C. to dissolve oxalic acid.

A mixture of 2-(3,3,5,5-tetramethylcyclohexyl)phenylamine (274.6 g, 1.09 mol) and ethyl acetate (1190 mL) was added dropwise to the solution of oxalic acid with keeping the temperature at around 50° C. over about 3.5 hours.

The vessel containing the solution of 2-(3,3,5,5-tetramethylcyclohexyl)phenylamine was washed with ethyl acetate (300 mL), and the mixture was cooled to about 20° C. and allowed to stand for about 3 hours.

The crystals were filtered, washed with ethyl acetate (600 mL) and dried under reduced pressure at about 50° C. to give the title compound (348.6 g, 99%) as white crystals.

The product was identical to the compound of Example 1-E by HPLC analysis.

Alternative method for Example 1-F: 1-[2-(3,3,5,5-tetramethylcyclohexyl)phenyl]piperazine methanesulfonate

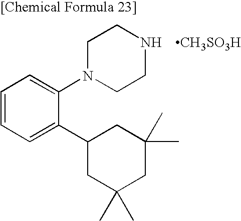

2-(3,3,5,5-Tetramethylcyclohexyl)phenylamine oxalate (338.6 g, 1.05 mol) and toluene (1700 mL) were placed in a 3 L separable flask, and a 1N aqueous solution of KOH (2415 g) was added, followed by stirring for 30 minutes. The mixture was allowed to stand, and the aqueous layer was discarded, and the organic layer was washed with water (1700 mL) and a 5% aqueous solution of sodium chloride in this order.

The organic layer was filtered and insoluble matter was washed with toluene (85 mL), concentrated under reduced pressure to give 2-(3,3,5,5-tetramethylcyclohexyl)phenylamine (244 g, 98.8%) as a pale brown oil.

2-(3,3,5,5-Tetramethylcyclohexyl)phenylamine (264.4 g, 1.098 mol), bis(2-chloroethyl)amine hydrochloride (333 g), N-methyl-2-pyrrolidone (1270 mL) and potassium carbonate (426 g) were placed in a 5 L 4-neck round bottom flask.

The mixture was rapidly heated to 170 to 180° C., and the temperature was kept for 7 hours, then the mixture was cooled down to 70° C.

Ethyl acetate (1270 mL) was added, and the reaction mixture was transferred to a 9 L separable flask, and water (1270 mL) was added with keeping the temperature at 50° C. or higher, and the reaction mixture was heated to about 80° C.

The reaction mixture was filtered using a filter pre-coated with celite, and the residue was washed with ethyl acetate (1270 mL), which was combined with the filtrate and liquid-liquid extraction was performed.

The organic layer was washed twice with water (1270 mL) and concentrated under reduced pressure.

The concentrate was placed in a 5 L separable flask, ethyl acetate (4320 mL) was added, and methanesulfonic acid (79.1 g) was added at about 30° C.

After allowing to stand for about 2 hour, the reaction mixture was filtered under reduced pressure, and dried under reduced pressure at about 80° C. to give the title compound (233 g, 53.4%) as crude crystals of pale brown solid.

Crude crystals of the title compound (236 g) and 2-propanol (1180 mL) were placed in a 5 L separable flask, and the mixture was heated to about 80° C., and ethyl acetate (2360 mL) was added dropwise over about 1 hour.

After allowing to stand at the same temperature for about 0.5 hours, the mixture was cooled down to about 25° C. and kept for 1 hour.

The crystals were filtered, washed with a mixed solution of 2-propanol (157 mL) and ethyl acetate (315 mL), and concentrated under reduced pressure at about 80° C. to give the title compound (214 g, 90.7%) as white crystals.

The product was identical to the compound of Example 1-F by HPLC analysis.

### Example 2

### 2-(3,3,5,5-Tetramethylcyclohexyl)amine

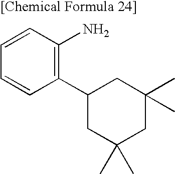

### Example 2-A

### N-(2-Bromophenyl)-2,2-dimethylpropionamide

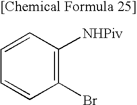

To a solution of 2-bromoaniline (30 g, 174 mmol) in toluene (300 mL) were added triethylamine (35.4 g, 348 mmol) and pivaloyl chloride (21.4 g, 178 mmol) at room temperature, followed by stirring at the same temperature for 2.5 hours. The reaction mixture was washed once with a 1N aqueous solution of hydrochloric acid and twice with a 5% aqueous solution of sodium chloride, dried over magnesium sulfate, filtered, and concentrated under reduced pressure. To the resultant crude crystals were added methanol (200 mL) and water (400 mL), which was heated at 100° C., and after confirming the dissolution of the crystals, the mixture was allowed to cool down to room temperature. Seed crystals (100 mg) were added to the mixture, followed by stirring at room temperature for 3 hours 40 minutes. The crystals were collected by filtration and dried to give 44.6 g of the title compound as white crystals.

¹H NMR (400 MHz, CDCl₃) δ: 1.35 (9H, s), 6.96 (1H, dt, J=2, 8 Hz), 7.31 (1H, dt, J=2, 8 Hz), 7.53 (1H, dd, J=2, 8 Hz), 8.01 (1H, brs, NH), 8.39 (1H, dd, J=2, 8 Hz).

### Example 2-B

### N-[2-(1-Hydroxy-3,3,5,5-tetramethylcyclohexyl)phenyl]-2,2-dimethylpropionamide

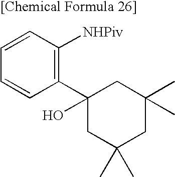

A solution of N-(2-bromophenyl)-2,2-dimethylpropionamide (3.9 g, 15.2 mmol) in tetrahydrofuran (39 mL) was cooled to −78° C. under nitrogen atmosphere, and to this solution was added dropwise n-butyllithium (2.71 M solution in hexane, 14 mL, 38 mmol) at −78° C. The reaction mixture was stirred at the same temperature for 25 minutes, a solution of 3,3,5,5-tetramethylcyclohexanone (5.34 mL, 30.4 mmol) in tetrahydrofuran (8 mL) was added dropwise, followed by stirring at the same temperature for 1 hour 30 minutes. Water (10 mL) was added to the reaction mixture at −78° C., extraction was carried out with ethyl acetate. The organic layer was washed with a 5% aqueous solution of sodium chloride, dried over magnesium sulfate, filtered, and concentrated.

The reaction was carried out using N-(2-bromophenyl)-2,2-dimethylpropionamide (1 g, 3.73 mmol) by the same procedures as described above, and the resultant crude product was combined.

Methanol (100 mL) was added to the combined crude crystals, which was heated and stirred, and water (25 mL) was added while heating. After dissolution of the mixture, the mixture was allowed to cool down to room temperature and stirred at the same temperature for 2 hours. The crystals were collected by filtration and dried to give 4.1 g of the title compound as white crystals.

¹H NMR (400 MHz, CDCl₃) δ: 0.96 (4.5H, s), 1.16-1.22 (1H, m), 1.33 (6H, s), 1.35 (4.5H, s), 1.46-1.52 (1H, m), 1.57 (6H, s), 1.60-1.71 (2H, m), 1.98-2.45 (2H, m), 7.02 (1H, dt, J=2, 8 Hz), 7.18-7.36 (3H, m), 8.34 (1H, J=8 Hz), 10.16 (1H, brs).

### Example 2-C

### N-[2-(3,3,5,5-Tetramethylcyclohex-1-enyl)phenyl]-2,2-dimethylpropionamide

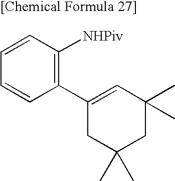

To a solution of N-[2-(1-hydroxy-3,3,5,5-tetramethylcyclohexyl)phenyl]-2,2-dimethylpropionamide (6.3 g, 19 mmol) in toluene (63 mL) was added pyridinium p-toluenesulfonate (477 mg, 1.9 mmol) at room temperature, followed by stirring at 90° C. for 2 hours. The reaction mixture was allowed to cool down to room temperature, and the organic layer was washed with water and a 5% aqueous solution of sodium chloride, dried over anhydrous magnesium sulfate, filtered, and concentrated to give 5.96 g of the title compound as a pale yellow solid.

¹H NMR (400 MHz, CDCl₃) δ: 1.06 (6H, s), 1.12 (6H, s), 1.28 (9H, s), 1.46 (2H, s), 2.00 (2H, s), 5.52 (1H, s), 7.01-7.06 (2H, m), 7.20-7.24 (1H, m), 7.94 (1H, brs), 8.37 (1H, d, J=8 Hz).

### Example 2-D

### N-[2-(3,3,5,5-Tetramethylcyclohexyl)phenyl]-2,2-dimethylpropionamide

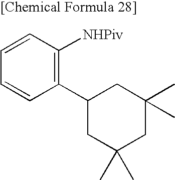

To a solution of N-[2-(3,3,5,5-Tetramethylcyclohex-1-enyl)phenyl]-2,2-dimethylpropionamide (5.7 g, 18.2 mmol) in ethanol (102 mL) was added 10% palladium on carbon (50% wet, 1.19 g), followed by stirring at room temperature, at atmospheric pressure under hydrogen atmosphere for 3 hours. The reaction mixture was filtered through celite and concentrated to give 5.74 g of the title compound as a pale yellow solid.

¹H NMR (400 MHz, CDCl₃) δ: 0.95 (6H, s), 1.12 (6H, s), 1.12-1.40 (4H, m), 1.35 (9H, s), 1.48-1.57 (2H, m), 2.99 (1H, tt, J=3, 12 Hz), 7.12-7.23 (3H, m), 7.24-7.30 (1H, m), 7.70 (1H, dd, J=2, 8 Hz)

### Example 2-E

### 2-(3,3,5,5-Tetramethylcyclohexyl)phenylamine

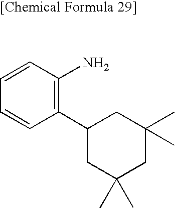

To a solution of N-[2-(3,3,5,5-Tetramethylcyclohexyl)phenyl]-2,2-dimethylpropionamide (330 mg, 1.05 mmol) in ethylene glycol (6 mL) was added sodium methoxide (28%, 6.04 mL, 31.4 mmol) at room temperature, followed by stirring at 160° C. for 8 hours and at room temperature for 15 hours 30 minutes. The reaction mixture was again stirred at 160° C. for 5 hours. The reaction mixture was allowed to cool down to room temperature, diluted with t-butyl methyl ether, washed with water and a 5% aqueous solution of sodium chloride, dried over anhydrous magnesium sulfate, filtered, and concentrated to give 243 mg of the title compound as a brown oil. Proton NMR spectrum corresponded with NMR spectrum for the title compound synthesized by the method of Example 1-D.

### Example 3

### 1-Cyclopropylmethyl-4-[2-(3,3,5,5-tetramethylcyclohexyl)phenyl]piperazine methanesulfonate

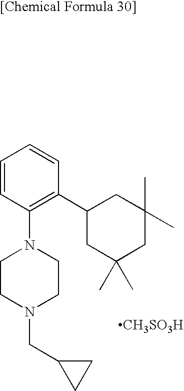

### Example 3-A

### Trifluoromethanesulfonic acid 3,3,5,5-tetramethylcyclohex-1-enyl Ester

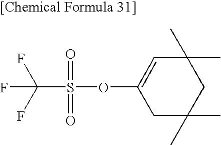

3,3,5,5-Tetramethylcyclohexanone (100.0 g, 648.3 mmol) was dissolved in anhydrous tetrahydrofuran (750 mL) under a nitrogen atmosphere, and the mixture was cooled and stirred at an external temperature of below −70° C. To the mixture was added dropwise lithium bis(trimethylsilyl)amide (1 M solution in tetrahydrofuran, 778 mL, 778 mmol) over a period of 30 minutes under the same conditions, followed by stirring for 70 minutes under the same conditions. Then, a solution of N-phenylbis(trifluoromethanesulfonimide) (254.8 g, 713 mmol) in anhydrous tetrahydrofuran (1 L) was added dropwise to the reaction mixture over a period of 35 minutes. After stirring the mixture for 20 minutes under the same conditions, the mixture was stirred for 15 hours while gradually warmed to an external temperature of room temperature. Reaction was repeated twice more on the same scale as above, by the same procedure under the same reaction conditions. The three reaction mixtures were combined and subjected to the following treatment.

Ethyl acetate (1.5 L) was added to the combined reaction mixture, and then a solution of concentrated hydrochloric acid (450 mL) in ice water (5 L) was added while stirring. After stirring for a while, the separated organic layer was washed with brine (1.5 L), a saturated aqueous solution of sodium hydrogencarbonate (1.5 L) and brine (1.5 L). The obtained organic layer was dried over anhydrous magnesium sulfate (1.5 kg) for 30 minutes while stirring. The desiccant was removed by filtration and the filtrate was concentrated under reduced pressure. The resultant residue was purified by silica gel column chromatography (hexane) and then dried under reduced pressure to give 520.94 g of the title compound as a light yellow oil.

¹H-NMR (400 MHz, CDCl₃) δ: 1.05 (s, 6H), 1.10 (s, 6H), 1.35 (s, 2H), 2.09 (d, J=1.2 Hz, 2H), 5.51 (t, J=1.2 Hz, 1H).

### Example 3-B

### 1-Nitro-2-(3,3,5,5-tetramethylcyclohex-1-enyl)benzene

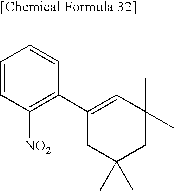

To a mixture of trifluoromethanesulfonic acid 3,3,5,5-tetramethylcyclohex-1-enyl ester (160.0 g, 558.8 mmol), 2-nitrophenylboronic acid (97.9 g, 586.8 mmol) and 1,2-dimethoxyethane (920 mL) were added sodium carbonate (118.5 g, 1.12 mol) and purified water (230 mL) while stirring at room temperature. Then, tetrakis(triphenylphosphine)palladium(0) (29.1 g, 25.1 mmol) was added to the mixture at room temperature (in an oil bath at room temperature), and the inside of the flask was replaced with nitrogen gas. The mixture was then stirred for 4 hours and 30 minutes at an external temperature of room temperature (in an oil bath at room temperature).

The same reaction was then repeated twice more by the same procedure under the same reaction conditions as above, but with an amount of 170.0 g (593.7 mmol) of trifluoromethanesulfonic acid 3,3,5,5-tetramethylcyclohex-1-enyl ester, a starting material, and the amounts of the other reagents changed to corresponding equivalents. The three reaction mixtures were combined and subjected to the following treatment.

Ethyl acetate (1.5 L) and water (4 L) were added to the combined reaction mixture, which was then stirred for 5 minutes. The mixture was filtered through Celite to remove insoluble materials. After stirring the obtained filtrate for a while, the organic layer was separated and the aqueous layer was extracted with ethyl acetate (1 L). The organic layers were combined and then dried over anhydrous magnesium sulfate (1 kg) for 20 minutes while stirring. The desiccant was removed by filtration and the filtrate was concentrated under reduced pressure. The resultant residue was purified by silica gel column chromatography (ethyl acetate/hexane) and then dried under reduced pressure to give 407.30 g of the title compound as a yellow solid.

¹H-NMR (400 MHz, CDCl₃) δ: 1.046 (s, 6H), 1.053 (s, 6H), 1.41 (s, 2H), 2.02 (d, J=1.6 Hz, 2H), 5.37 (t, J=1.6 Hz, 1H), 7.26 (dd, J=7.6, 1.6 Hz, 1H), 7.33 (ddd, J=8.0, 7.6, 1.6 Hz, 1H), 7.49 (ddd, J=7.6, 7.6, 1.2 Hz, 1H), 7.74 (dd, J=8.0, 1.2 Hz, 1H).

### Example 3-C

### 2-(3,3,5,5-Tetramethylcyclohexyl)phenylamine

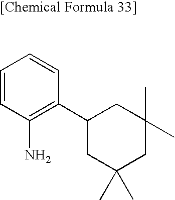

A mixture of 1-nitro-2-(3,3,5,5-tetramethylcyclohex-1-enyl)benzene (130.0 g, 501.3 mmol), 10% palladium on carbon (13.0 g, wet) and ethyl alcohol (1820 mL) was placed in a flask, then the inside of the flask was replaced with hydrogen, and the mixture was stirred for 78 hours at room temperature under a hydrogen atmosphere at atmospheric pressure. Reaction was repeated two more times on the same scale as above, by the same procedure under the same reaction conditions. The three reaction mixtures were combined and subjected to the following treatment.

The combined reaction mixture was filtered, and the filtrate was concentrated under reduced pressure. The obtained residue was diluted with ethyl acetate (700 mL) and hexane (200 mL), and then dried over anhydrous sodium sulfate (200 g) for 20 minutes while stirring. The desiccant was removed using a glass microfibre filter, and then the filtrate was concentrated and dried under reduced pressure to give 345.76 g of the title compound as a light brown oil.

¹H-NMR (400 MHz, CDCl₃) δ: 0.95 (s, 6H), 1.13 (s, 6H), 1.08-1.36 (m, 4H), 1.59-1.62 (m, 2H), 2.86 (tt, J=12.4, 2.8 Hz, 1H), 3.63 (brs, 2H), 6.70 (dd, J=7.6, 1.2 Hz, 1H), 6.78 (ddd, J=7.6, 7.6, 1.2 Hz, 1H), 7.02 (ddd, J=7.6, 7.6, 1.2 Hz, 1H), 7.12 (dd, J=7.6, 1.2 Hz, 1H).

### Example 3-D

### 1-[2-(3,3,5,5-Tetramethylcyclohexyl)phenyl]piperazine

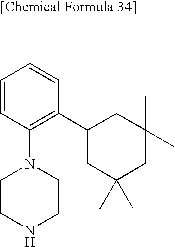

To a mixture of 2-(3,3,5,5-tetramethylcyclohexyl)phenylamine (168.0 g, 726.1 mmol) and 1,2-dichlorobenzene (1200 mL) was added bis(2-chloroethyl)amine hydrochloride (155.5 g, 871.3 mmol). The mixture was stirred for 7 hours at an external temperature of 190° C. under a nitrogen atmosphere. During the reaction, a nitrogen stream was passed through the reactor several times to remove the generated hydrogen chloride gas. Reaction was repeated once more on the same scale as above, by the same procedure under the same reaction conditions. The two reaction mixtures were combined and subjected to the following treatment.

After cooling to room temperature, the combined reaction mixture was diluted with ethyl acetate (6 L) and water (1 L). The mixture was then added to a mixture of potassium carbonate (1.3 kg) and water (5 L) while stirring. The mixture was stirred and allowed to stand, and the organic layer was separated. The aqueous layer was again extracted with ethyl acetate (2 L). The combined organic layers were washed with brine (3 L) and then dried over anhydrous sodium sulfate (3.5 kg). The desiccant was removed by filtration and the filtrate was concentrated under reduced pressure. The resultant residue was purified by NH silica gel column chromatography (ethyl acetate/hexane) and then dried under reduced pressure to give 241.67 g of the title compound as a light pink solid.

In addition to this, the above NH silica gel column chromatography purification also yielded 126.2 g of an oil as a mixture of the target compound and impurities. Hexane (150 mL) was added to the oil, and the mixture was stirred for 2 hours at 0° C. The produced precipitate was collected by suction filtration and then dried under reduced pressure to give 42.74 g of the title compound as a light pink solid. A total of 284.41 g of the title compound was obtained as a light pink solid.

¹H-NMR (400 MHz, CDCl₃) δ: 0.93 (s, 6H), 1.13 (s, 6H), 1.17-1.35 (m, 4H), 1.42-1.46 (m, 2H), 2.84-2.87 (m, 4H), 3.02-3.04 (m, 4H), 3.60 (tt, J=12.8, 2.8 Hz, 1H), 7.06-7.18 (m, 3H), 7.23 (dd, J=7.6, 1.6 Hz, 1H). The 1H of NH could not be identified.

### Example 3-F

### 1-Cyclopropylmethyl-4-[2-(3,3,5,5-tetramethylcyclohexyl)phenyl]piperazine

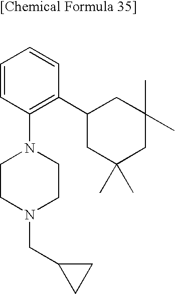

To a mixture of 1-[2-(3,3,5,5-tetramethylcyclohexyl)phenyl]piperazine (241.67 g, 804.3 mmol), acetic acid (46.0 mL, 804.3 mmol) and tetrahydrofuran (3300 mL) was added a mixed solution of cyclopropanecarbaldehyde (64.8 g, 924.9 mmol) and tetrahydrofuran (200 mL) while stirring at an external temperature of room temperature. After stirring for 10 minutes, sodium triacetoxyborohydride (238.6 g, 1126 mmol) was added portionwise to the reaction mixture over a period of 8 minutes. The mixture was then stirred for 3 hours at an external temperature of room temperature.

The reaction mixture was diluted with hexane (2 L) and water (1 L). This mixture was then added to a mixture of potassium carbonate (667 g) and water (3.5 L) while stirring. After stirring for a while and allowing the mixture to stand, the separated organic layer was washed sequentially with water (2 L) and brine (1.5 L). The organic layer was then dried over anhydrous sodium sulfate (1.5 kg), the desiccant was removed by filtration, and the obtained filtrate was concentrated under reduced pressure. The obtained residue was purified by NH silica gel column chromatography (ethyl acetate/hexane) and then concentrated under reduced pressure to give an oil. The oil was dissolved again in ethyl acetate (1 L), and the mixture was filtered through a glass microfibre filter to remove insoluble materials. The obtained filtrate was concentrated under reduced pressure, and then a vacuum pump was used for drying under reduced pressure for 2 hours at an external temperature of 50° C., to give 280.7 g of the title compound as crystals.

¹H-NMR (400 MHz, CDCl₃) δ: 0.12-0.16 (m, 2H), 0.52-0.56 (m, 2H), 0.88-0.96 (m, 1H), 0.92 (s, 6H), 1.12 (s, 6H), 1.13-1.34 (m, 4H), 1.41-1.47 (m, 2H), 2.32 (d, J=6.4 Hz, 2H), 2.40-2.98 (br, 4H), 2.94-2.96 (m, 4H), 3.58 (tt, J=12.6, 2.8 Hz, 1H), 7.05-7.18 (m, 3H), 7.22-7.24 (m, 1H).

### Example 3-G

### 1-Cyclopropylmethyl-4-[2-(3,3,5,5-tetramethylcyclohexyl)phenyl]piperazine methanesulfonate

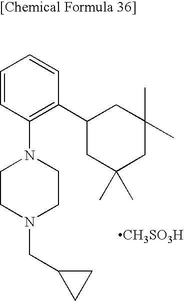

A mixture of 1-cyclopropylmethyl-4-[2-(3,3,5,5-tetramethylcyclohexyl)phenyl]piperazine (277.0 g, 781.2 mmol) and 2-butanone (2493 mL) was stirred while heating at an external temperature of 81° C. Methanesulfonic acid (76.58 g, 796.8 mmol) was then added dropwise thereto over a period of 3 minutes, to form a thoroughly dissolved state. After heating and stirring for 7 minutes at an external temperature of 81° C., the external temperature was gradually lowered and stirring was continued until the internal temperature reached 37° C. The reaction suspension containing the produced precipitate was transferred to another flask using 2-butanone (100 mL). The suspension was then concentrated under reduced pressure over a period of 1 hour and 20 minutes at an external temperature of 21° C. It was then dried under reduced pressure for 30 minutes at an external temperature of 40° C. for solidification of the flask contents, to give a crude solid product of the title compound. After adding a mixed solvent of ethyl acetate (1662 mL) and heptane (1108 mL) to this crude solid product, the resulting suspension was stirred for 1 hour at an external temperature of 65° C. The suspension was then further stirred while gradually lowering the external temperature, and after the external temperature reached 45° C., stirring was continued for 14 hours at an external temperature of room temperature. The obtained suspension was filtered and the precipitated solid was collected. The solid was washed with a mixed solvent of ethyl acetate (330 mL) and heptane (220 mL) and aircured by aspiration for 4 hours at room temperature. The obtained crystals were dried for 6 hours at 70° C. in a warm-air drier to give 335.9 g of the title compound as colorless (white) powdery crystals (crystal a).

¹H-NMR (400 MHz, CDCl₃) δ: 0.47-0.51 (m, 2H), 0.81-0.85 (m, 2H), 0.94 (s, 6H), 1.10 (s, 6H), 1.15-1.43 (m, 7H), 2.85 (s, 3H), 2.95-3.11 (m, 6H), 3.43 (tt, J=12.6, 3.0 Hz, 1H), 3.52-3.61 (m, 2H), 3.80 (br d, J=11.2 Hz, 2H), 7.13-7.26 (m, 4H), 11.11 (br s, 1H).

### Example 4

### 3-[2-(1-Hyrdoxy-3,3,5,5-tetramethylcyclohexyl)phenyl]1,1-dimethylurea

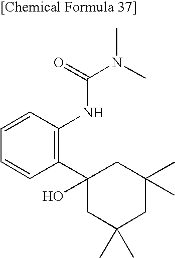

The title compound was obtained according to the similar methods to Example 2-B using 3-(2-bromophenyl)-1,1-dimethylurea instead of N-(2-bromophenyl)-2,2-dimethylpropionamide.

¹H-NMR (400 MHz, CDCl₃) δ: 0.91 (6H, s), 1.12-1.48 (4H, m), 1.28 (6H, s), 1.92-1.98 (2H, m), 2.94 (6H, s), 5.91 (1H, s), 6.89 (1H, t, J=8 Hz), 7.15 (1H, t, J=8 Hz), 7.23 (1H, d, J=8 Hz), 8.09 (1H, d, J=8 Hz), 10.01 (1H, s).

### Example 5

### 4-[2-(3,3,5,5-tetramethylcyclohex-1-enyl)phenyl]piperazine-1-carboxylic Acid Tert-Butyl Ester

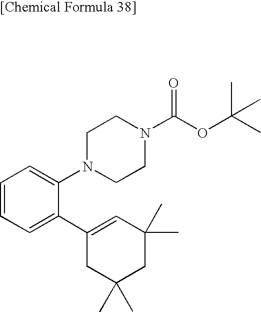

### Example 5-A

### 1-Bromo-2-(3,3,5,5-tetramethyl-1-cyclohex-1-enyl)benzene

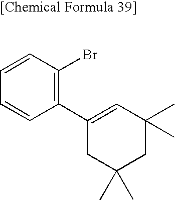

Tetrakis(triphenylphosphine)palladium (79 mg, 0.0686 mmol) and a 2N aqueous solution of sodium carbonate (2.5 mL) was added to a solution of 2-bromophenylboronic acid (689 mg, 3.43 mmol) and 3,3,5,5-tetramethyl-1-cyclohexenyl trifluoromethanesulfonate (655 mg, 2.29 mmol) in toluene (14 mL) at room temperature under nitrogen atmosphere, followed by stirring under reflux for 4 hours. The reaction mixture was cooled down to room temperature, extracted with ethyl acetate, washed with brine, dried over sodium sulfate, filtered, and concentrated. The resultant crude product was purified with silica gel column chromatography (n-hexane/ethyl acetate=100/1) to give 1-bromo-2-(3,3,5,5-tetramethyl-1-cyclohex-1-enyl)benzene (390 mg, 39%) as a colorless oil.

¹H NMR (400 MHz, CDCl₃) δ 1.06 (6H, s), 1.07 (6H, s), 1.41 (2H, s), 2.04 (2H, s), 5.39 (1H, s), 7.03-7.33 (3H, m), 7.53 (1H, m).

### Example 5-B

### 4-[2-(3,3,5,5-tetramethylcyclohex-1-enyl)phenyl]piperazine-1-carboxylic acid tert-butyl ester

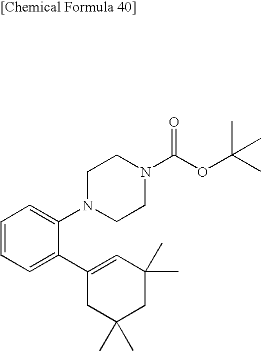

A solution of 1-bromo-2-(3,3,5,5-tetramethyl-1-cyclohex-1-enyl)benzene (20 mg, 0.0682 mmol), N-tert-butoxycarbonylpiperazine (19 mg, 0.102 mmol), palladium acetate (2 mg, 0.0068 mmol), 2,2′-bis(diphenylphosphino)-1,1′-binaphthyl (17 mg, 0.0273 mmol) and sodium tert-butoxide (10 mg, 0.102 mmol) in toluene (0.25 mL) was heated up to 100° C. and stirred for 4 hours under nitrogen atmosphere. The whole reaction mixture was purified by thin layer chromatography (n-hexane/ethyl acetate=3/1) to give 4-[2-(3,3,5,5-tetramethylcyclohex-1-enyl)phenyl]piperazine-1-carboxylic acid tert-butyl ester (14 mg, 52%) as a colorless oil.

¹H NMR (400 MHz, CDCl₃) δ 1.02 (6H, s), 1.07 (6H, s), 1.39 (2H, s), 1.49 (9H, s), 2.16 (2H, s), 2.91 (4H, m), 3.51 (4H, m), 5.50 (1H, s), 6.97 (1H, m), 7.00 (1H, m), 7.08 (1H, m), 7.19 (1H, m).

### Example 6

### 4-[2-(3,3,5,5-tetramethylcyclohex-1-enyl)phenyl]piperazine-1-carboxylic Acid Tert-Butyl Ester

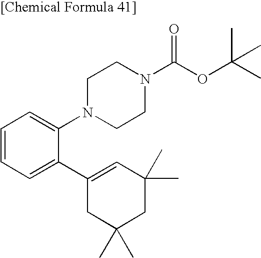

### Example 6-A

### 2-[(4-tert-butoxycarbonyl)piperazin-1-yl]phenylboronic Acid

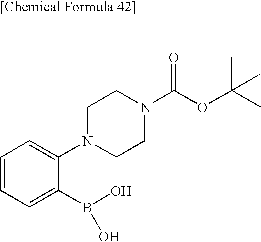

A 1.6 M solution of n-butyllithium in hexane (2.11 mL, 3.37 mmol) was added dropwise to a solution of 4-(2-bromophenyl)piperazine-1-carboxylic acid tert-butyl ester (1.0 g, 2.93 mmol) in dehydrated tetrahydrofuran (10 mL) at −70° C. under nitrogen atmosphere, followed by stirring at the same temperature for 1 hour. Triisopropyl borate (0.88 mL, 3.81 mmol) was added to the reaction mixture at −70° C., followed by stirring at the same temperature for 10 minutes. The reaction mixture was brought to room temperature and stirred for 1 hour, and isopropyl alcohol (8 mL) and a saturated aqueous solution of ammonium chloride (8 mL) to the reaction mixture, followed by vigorous stirring for 30 minutes. To the residue obtained by concentrating the mixture under reduced pressure was added a saturated aqueous solution of sodium chloride, and extraction was carried out with ethyl acetate. The organic layer was dried over anhydrous sodium sulfate, and filtered, and the filtrate was concentrated under reduced pressure. n-Hexane (30 mL) was added to the resultant pale yellow oil, and the mixture was subjected to sonication to precipitate crystals. The crystals were collected by filtration and dried under reduced pressure to give the title compound (715 mg) as white crystals.

¹H NMR (400 MHz, CDCl₃) δ 1.49 (9H, s), 2.90-2.96 (4H, m), 3.60-3.67 (4H, m), 7.23-7.27 (2H, m), 7.43-7.48 (1H, m), 7.63 (2H, brs), 7.89-7.92 (1H, m).

### Example 6-B

### 4-[2-(3,3,5,5-Tetramethylcyclohex-1-enyl)phenyl]piperazine-1-carboxylic Acid Tert-Butyl Ester

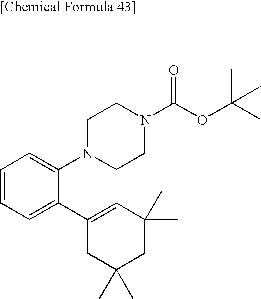

A solution of 2-[(4-tert-butoxycarbonyl)piperazin-1-yl]phenylboronic acid (47.9 mg, 0.157 mmol), 3,3,5,5-tetramethyl-1-cyclohexenyl trifluoromethanesulfonate (50 mg, 0.174 mmol), palladium acetate (1.96 mg, 0.0087 mmol), 2-dicyclohexylphosphino-2′,6′-diethoxybiphenyl (21.4 mg, 0.0522 mmol) and cesium fluoride (79.3 mg, 0.522 mmol) in dimethoxyethane (1 mL) and water (100 μL) was heated up to 50° C. and stirred for 4 hours under nitrogen atmosphere. The reaction mixture was diluted with ethyl acetate, washed twice with water, and dried over magnesium sulfate. The desiccant was removed by filtration, and the black residue obtained by distilling off the solvent was purified by silica gel chromatography (n-hexane/ethyl acetate=9/1) to give 4-[2-(3,3,5,5-tetramethylcyclohex-1-enyl)phenyl]piperazine-1-carboxylic acid tert-butyl ester (23 mg, 37%) as a colorless oil. NMR data for the product corresponded with that for the compound obtained in Example 5.

¹H NMR (400 MHz, CDCl₃) δ 1.02 (6H, s), 1.07 (6H, s), 1.39 (2H, s), 1.49 (9H, s), 2.16 (2H, s), 2.91 (4H, m), 3.51 (4H, m), 5.50 (1H, s), 6.97 (1H, m), 7.00 (1H, m), 7.08 (1H, m), 7.19 (1H, m).

(Evaluation of Compound)

1-Cyclopropylmethyl-4-[2-(3,3,5,5-tetramethylcyclohexyl)phenyl]piperazine methanesulfonate (Compound (B)) was evaluated according to the Test Example 1 to 4 below, and it showed a good activity.

### Test Example 1

### Evaluation of Compounds in Jurkat Cell Adhesion System

### <Immobilization of Human Fibronectin in 96-Well Plate>

Human fibronectin (Becton Dickinson Biosciences) was diluted with phosphate-buffered saline (hereinafter abbreviated as PBS; Sigma) to 0.1-0.01 μg/mL, and the diluted solution was added to a 96-well plate (Becton Dickinson) at 50 μL/well, and allowed to stand overnight at 4° C. On the following day, the supernatant was removed from the plate, and then PBS containing 1% bovine serum albumin (hereinafter abbreviated as BSA; Sigma) was added thereto at 100 μL/well and incubation was performed at 37° C. for 2 hours in a CO₂ incubator (Hirasawa).

<Adhesion Assay>

The supernatant was removed from each plate and Jurkat cells suspended in RPMI-1640 (Sigma) containing 1 mg/mL BSA were added at 80 μL/well for 2.5×10⁵ cells/well. The compound (B) diluted to different concentrations with RPMI-1640 containing 1 mg/nl BSA was immediately added at 10 μL/well, and then 100 nM phorbol myristate acetate (hereinafter abbreviated as PMA; Sigma) in RPMI-1640 containing 1 mg/mL BSA was added at 10 μL/well and the plate was incubated in a CO₂ incubator at 37° C. for 45-60 minutes. The supernatant was removed from the plate and each well was washed several times with 100 μL/well of RPMI-1640, after which 50 mM citrate buffer (pH 5.0) containing 3.75 mM p-nitrophenol-N-acetyl-β-D-glucosaminide (Sigma) and 0.25% Triton X-100 (Sigma) were added at 60 μL/well, and the mixture was placed in a CO₂ incubator and incubated at 37° C. for 45 minutes. After incubation, 50 mM glycine buffer (pH 10.4) containing 5 mM EDTA was added at 90 μL/well, and the absorbance at 405 nm was measured with an EL340 Automated Microplate Reader (BIO-TEK) to determine the adhered cell count. IC₅₀ (concentration which inhibited the increase in the number of adhered cells by the PMA-stimulation by 50%) for compound (B) was 4.7 μM.

### Test Example 2

### Evaluation of Compounds in Human Peripheral Blood Neutrophil Adhesion System

### <Preparation of Human Peripheral Blood Neutrophils>

To a plastic centrifugation tube containing 100 units of heparin sodium (Shimizu Pharmaceutical) was added 25 mL of fresh blood sampled from a healthy human. After adding and mixing therewith 8 mL of physiological saline (Otsuka Pharmaceutical) containing 6% Dextran (Nacalai), the mixture was allowed to stand at room temperature for 45 minutes for sedimentation of the erythrocytes. The resultant supernatant was transferred to another plastic centrifugation tube and combined with an equivalent volume of PBS, and then centrifuged at 1600 rpm for 7 minutes at room temperature. The obtained hematocyte fraction was suspended in 4 mL of PBS, and the suspension was superposed on 4 mL of Ficoll-Paque™ PLUS (Amersham Biosciences). The resultant bilayer liquid was centrifuged at 2000 rpm for 30 minutes at room temperature, after which the supernatant was removed and the precipitate was suspended in 10 mL of PBS and centrifuged at 1200 rpm for 7 minutes, and the supernatant was removed. The resulting precipitate was suspended in 0.5 mL of PBS again, and then 10 mL of distilled water (Otsuka Pharmaceutical) was added, 0.5 mL of an aqueous solution containing 3 M NaCl was immediately added to restore isotonicity, the mixture was centrifuged at 1200 rpm for 7 minutes, and the obtained precipitate was suspended in PBS containing 1 mg/mL BSA again and stored in ice until being used for the experiment.

<Fluorescent Labeling of Human Peripheral Blood Neutrophils>

The obtained neutrophils were suspended in PBS containing 1 mg/mL BSA at 2×10⁷ cells/mL. BCECF-AM (Dojin) was added to a final concentration of 5 μM, and the mixture was incubated at 37° C. for 45 minutes. It was then rinsed twice with PBS containing 1 mg/mL BSA by centrifugation, suspended again in PBS containing 1 mg/mL BSA at 5×10⁷ cells/mL, and stored in ice until use.

<Preparation of HUVEC Immobilized Plate>

Human umbilical vein endothelial cells (hereinafter abbreviated as HUVEC) were suspended in MCDB131 medium (Chlorella Industries) containing 10% fetal calf serum and 30 μg/mL endothelial cell growth supplement (Becton Dickinson Bioscience). The suspension was added at 7.5×10³ cells/well to a 96-well plate (Iwaki) immobilized with type I collagen, and cultured for 3 days in a CO₂ incubator (Hirasawa). Upon confirming confluency of the cells, the supernatant was discarded, the plate was rinsed twice with PBS, and then PBS containing 0.1% glutaraldehyde (Kanto Kagaku) was added at 100 μL/well and the HUVECs were immobilized for 5 minutes. The supernatant was discarded and the plate was washed twice with PBS, and then PBS was added at 100 μL/well and the mixture was stored at 4° C. until use.

<Adhesion Assay>

To 6.5 mL of RPMI-1640 medium (Sigma) containing 1 mg/mL of BSA were added 0.5 mL of a suspension of BCECF-AM labeled neutrophils at 5×10⁷ cells/mL stored in ice, which was mixed, and the mixture was added at 80 μL/well to a HUVEC immobilized plate. To this plate were immediately added 10 μL/well of a solution of the compound diluted at different concentrations with RPMI-1640 containing 1 mg/mL BSA, and 10 μL/well of 100 nM PMA in RPMI-1640 containing 1 mg/mL BSA, and the mixture was incubated in a CO₂ incubator at 37° C. for 45 minutes. The supernatant was removed from the plate, which was then washed several times with RPMI-1640 at 100 μL/well, and then PBS containing 0.1% NP-40 (Calbiochem) was added thereto at 100 μL/well and the fluorescent intensity was measured with an ARVO™SX 1420 multi label counter (Wallac) to determine the number of adhered cells. IC₅₀ (concentration which inhibited the increase in the number of adhered cells by the PMA-stimulation by 50%) for compound (B) was 7.1 μM.

### INDUSTRIAL APPLICABILITY

[2-(3,3,5,5-Tetramethylcyclohexyl)phenyl]piperazine compounds have excellent cell adhesion inhibitory action or cell infiltration inhibitory action, the compound of the invention can be useful intermediates in production of the [2-(3,3,5,5-tetramethylcyclohexyl)phenyl]piperazine compounds.

1. A compound represented by the following general formula (A) or salt thereof: 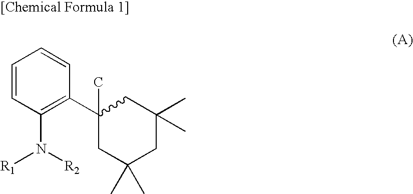
wherein R¹ and R² independently represent hydrogen or a protecting group for an amino group, or R¹ and R² may bond together to form an optionally substituted pyrrole ring, an optionally substituted piperidine ring or an optionally substituted piperazine ring,
the bond (B) represented by the following formula represents a single bond or a double bond
[Chemical Formula 2] [symbol]
(B)
and C represents hydroxyl or hydrogen when the bond (B) represents a single bond and C is absent when the bond (B) represents a double bond.

2. The compound or salt thereof according to claim 1, wherein the compound is represented by the general formula (A-1): 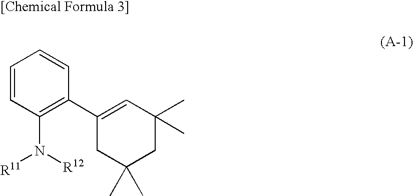
wherein R¹¹ and R¹² independently represent hydrogen or a protecting group for an amino group, or R¹¹ and R¹² may bond together to form an optionally substituted pyrrole ring or an optionally substituted piperidine ring.

3. The compound or salt thereof according to claim 1, wherein the compound is represented by the general formula (A-2): 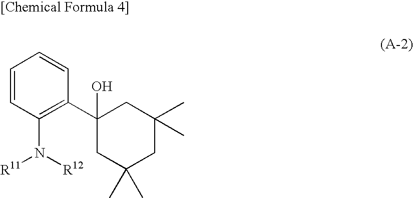
wherein R¹¹ and R¹² independently represent hydrogen or a protecting group for an amino group, or R¹¹ and R¹² may bond together to form an optionally substituted pyrrole ring or an optionally substituted piperidine ring.

4. The compound or salt thereof according to claim 1, wherein the compound is represented by the formula (A-3):

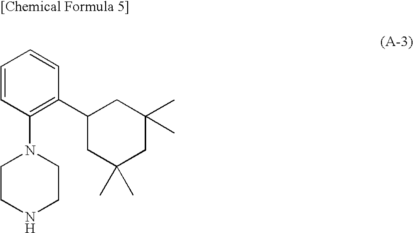

5. The compound or salt thereof according to any of claims 1 to 3, wherein the protecting group for an amino group is formyl, pivaloyl, trifluoroacetyl, trichloroacetyl, acetyl, phenylacetyl, benzoyl, N,N-dimethylaminocarbonyl, N-[2-trimethylsilylethoxy]methyl, t-butyloxycarbonyl, methyloxycarbonyl, ethyloxycarbonyl, 2,2,2-trichloroethyloxycarbonyl, 2-trimethylsilyloxycarbonyl, vinyloxycarbonyl, allyloxycarbonyl, benzyloxycarbonyl, p-methoxybenzyloxycarbonyl, p-nitrobenzyloxycarbonyl or benzyl.

6. The compound or salt thereof according to claim 1, wherein R² is hydrogen.

7. The compound or salt thereof according to claim 1, wherein R² is hydrogen and R¹ is hydrogen, formyl or pivaloyl.

8. The compound or salt thereof according claim 2, wherein R¹² is hydrogen.

9. The compound or salt thereof according to claim 2 or 3, wherein R¹² is hydrogen and R¹¹ is hydrogen, formyl or pivaloyl.

10. A salt of the compound according to any of claims 1 to 4 with an acid selected from oxalic acid, fumaric acid, p-toluenesulfonic acid or methanesulfonic acid.

11. A salt of the compound according to any of claims 1 to 4 with methanesulfonic acid.
